SEQUENCE LISTING (52 sequences)

SEQ ID NO: 1 - DNA, 303 bp, Homo sapiens
gaggtaatgaatgtcattgttttacagtttattcttcttgtgtttcttcttgtgaagatatataagcatgcagacacactattttatatatatatacctatatatgtatgtatgtgtatacacagttatgcactgtataacagtattttggtcagtgatggactgcgtatgctaaggtgttcccataagattataataagtactttgactataacttttctatgtttacatgcagaaatacttactaatgggttacagttgcctacagtattcagtacaccagcatgctgtacaggttggtag

SEQ ID NO: 2 - protein, 98 aa, Homo sapiens
MNVIVLQFILLVFLLVKIYKHADTLFYIYIPIYVCMCIHSYALYNSILVSDGLRMLRCSHKIIISTLTITFLCLHAEILTNGLQLPTVFSTPACCTGW

SEQ ID NO: 3 - DNA, 336 bp, Homo sapiens
agcattatggttattccacatcttgttttattgactttgatttcctttagattaaaagaaaaaaatagtgtatttcatttaattttccccgctattcacagtttatgcttatgtgattctggaagaattccagctaggaatgccttggacccatcccaggaccagcaacccctgcagcaggacaaagatggcactgagactatgtgtgtagctggaagcaacctaaatgtccattcgtgggtgaatgaagaaagaaaatgtggcatatccatacaatgtaatatcattcagcctttacaaagtaggaaactctgccgtttgctgcaacagagatga

SEQ ID NO: 4 - protein, 109 aa, Homo sapiens [sequence omitted]

SEQ ID NO: 5 - DNA, 330 bp, Homo sapiens
ggaagtatgagtcctagtttattcttcatttttaagattgttttggctattgtggattccctgcaattctatatgaatttcgaatcagtttgtcgatgtctgcaaaaaatctctgtgattctgatagggattgcttttaacctgtgtaacgatttggggagtattgtcattttaacagtgttatgtattctaatccatgaatatgaaatatatttccttttgtttagatctttgattttttcattatgttttatagttccagagtatagtaagttttgcaatttttatgttaaatttattcttaagaatttatttttgatgctatcataa

SEQ ID NO: 6 - protein, 107 aa, Homo sapiens
MSPSLFFIFKIVLAIVDSLQFYMNFESVCRCLQKISVILIGIAFNLCNDLGSIVILTVLCILIHEYEIYFLLFRSLIFSLCFIVPEYSKFCNFYVKFILKNLFLMLS

SEQ ID NO: 7 - DNA, 315 bp, Homo sapiens
tctaggatggtaactcctatctggacacttttcatttgttactgtttgacctctttgcttgtattacaggctatatttaaagaaatagataacattctctctgaggttgatttaaaccaacatcctgtacgttgctgctatagcttcccaacattttgtgtagaggggatgctattgaagttgtgttttaatatggagccacactgttttctttctctgacccagtctacagtcagcctgtcccaaggctgccatctattctctgtgtttgtgcagctcatctggacagctcatctggacagacacaaagaatag

SEQ ID NO: 8 - protein, 102 aa, Homo sapiens
MVTPIWTLFICYCLTSLLVLQAIFKEIDNILSEVDLNQHPVRCCYSFPTFCVEGMLLKLCFNMEPHCFLSLTQSTVSLSQGCHLFSVFVQLIWTAHLDRHKE

SEQ ID NO: 9 - DNA, 312 bp, Homo sapiens
tgtaatatgttatttactttattattccgaattctaatcggttatgtgagaactctgtggacgaaaaattcttgctgctgttggcgaatgattttaaatcattcatttaaacaagaagtgcctatgattgtagagctaaagcaaaaatgtgaaatgttttgtcagaaatatctagttgataaagattattcctttcgtgtttctgtaacctgtcagttctttatacttttacatgattcctacccaactgagaatacatggtcaactattccaacattgtctgctcttatatcctctttgatttttctgtga

SEQ ID NO: 10 - protein, 101 aa, Homo sapiens [sequence omitted]

SEQ ID NO: 11 - DNA, 345 bp, Homo sapiens
ccagacatgatctttggcctgctaatcaaagctctttatctagcgtcagcctgggcaggggctctgagcctcggcgctgctggcatttggggctggatgactctttgctgtggctgctgtcctgtgcattacaggacattacgtagcatccctgaccacaacctactagatgccagtagcaccccctccctagttatgacaaccagaaacatctccagacattgccaatgtcccctggtggcaaaatcatccccggctgagaatgagtgttgcacggtaattcctccattccaaattaacagagcacttaggaacgagtgctttctcctattactttccctttaa

SEQ ID NO: 12 - protein, 112 aa, Homo sapiens
MIFGLLIKALYLASAWAGALSLGAAGIWGWMTLCCGCCPVHYRTLRSIPDHNLLDASSTPSLVMTTRNISRHCQCPLVAKSSPAENECCTVIPPFQINRALRNECFLLLLSL

SEQ ID NO: 13 - DNA, 390 bp, Homo sapiens
tgaactatgggctttgttgttctatgcctaattttcttcctgtgtaagactggaatggattccagatttcaactaaaactcttgtttcactgttttcaaggacttttccaaaggtcacacatggactattgtgatgaatgcactctgcagggtgtgttcccagagcacagaagtaaccagagagctgcaagggaggtgttgcccacaccaaaacactgcagacttattcccctggggacagtgctgtcagagtgtccatttcaagctccctgttggccacagacaaaagccattatcctaaatctctggcgaaacttggaggtcttagaagtggacagaagtttaagacaggattgctttaaatgcacaattttgttagaatttttctga

SEQ ID NO: 14 - protein, 127 aa, Homo sapiens [sequence omitted]

SEQ ID NO: 15 - DNA, 282 bp, Homo sapiens
ctgactatgatgagggtgctaaggctgctggcgagggtcctcctcggccagcttctcctagcagcagggcacgcacagccctgttttctcatctgctttcagcagcatttgcctcctactccacttgggtcactcaagggtcccaaaatagacctgtgcattcatgggacccctcccacctgcctctctgctcagtgtctctgttgggacaggcagcaagtgcttaaatcccagccactgctccccgctggagtccacctgagaactttcccagcaatttga

SEQ ID NO: 16 - protein, 91 aa, Homo sapiens [sequence omitted]

SEQ ID NO: 17 - DNA, 21 bp, synthetic construct
atgaatgtcattgttttacag

SEQ ID NO: 18 - DNA, 20 bp, synthetic construct
ctaccaacctgtacagcatg

SEQ ID NO: 19 - DNA, 25 bp, synthetic construct
ctgactatgatgagggtgctaaggc

SEQ ID NO: 20 - DNA, 25 bp, synthetic construct
tcaaattgctgggaaagttctcagg

SEQ ID NO: 21 - DNA, 22 bp, synthetic construct
catgatctttggcctgctaatc

SEQ ID NO: 22 - DNA, 20 bp, synthetic construct
accgtgcaacactcattctc

SEQ ID NO: 23 - DNA, 22 bp, synthetic construct
ctatgggctttgttgttctatg

SEQ ID NO: 24 - DNA, 23 bp, synthetic construct
tcagaaaaattctaacaaaattg

SEQ ID NO: 25 - DNA, 19 bp, synthetic construct
atggttattccacatcttg

SEQ ID NO: 26 - DNA, 21 bp, synthetic construct
tcatctctgttgcagcaaacg

SEQ ID NO: 27 - DNA, 21 bp, synthetic construct
atgttatttactttattattc

SEQ ID NO: 28 - DNA, 20 bp, synthetic construct
tcacagaaaaatcaaagagg

SEQ ID NO: 29 - DNA, 37 bp, synthetic construct
aagcaggcttcgccaccatgaatgtcattgttttaca

SEQ ID NO: 30 - DNA, 32 bp, synthetic construct
gtgatggtgatggtgccaacctgtacagcatg

SEQ ID NO: 31 - DNA, 42 bp, synthetic construct
aagcaggcttcgccaccctgactatgatgagggtgctaaggc

SEQ ID NO: 32 - DNA, 37 bp, synthetic construct
gtgatggtgatggtgaattgctgggaaagttctcagg

SEQ ID NO: 33 - DNA, 39 bp, synthetic construct
aagcaggcttcgccacccatgatctttggcctgctaatc

SEQ ID NO: 34 - DNA, 33 bp, synthetic construct
gtgatggtgatggtgaagggaaagtaataggag

SEQ ID NO: 35 - DNA, 39 bp, synthetic construct
aagcaggcttcgccaccctatgggctttgttgttctatg

SEQ ID NO: 36 - DNA, 35 bp, synthetic construct
gtgatggtgatggtggaaaaattctaacaaaattg

SEQ ID NO: 37 - DNA, 36 bp, synthetic construct
aagcaggcttcgccaccatggttattccacatcttg

SEQ ID NO: 38 - DNA, 33 bp, synthetic construct
gtgatggtgatggtgtctctgttgcagcaaacg

SEQ ID NO: 39 - DNA, 38 bp, synthetic construct
aagcaggcttcgccaccatgttatttactttattattc

SEQ ID NO: 40 - DNA, 32 bp, synthetic construct
gtgatggtgatggtgcagaaaaatcaaagagg

SEQ ID NO: 41 - DNA, 57 bp, synthetic construct
ggggacaagtttgtacaaaaaagcaggcttcgccaccatgaatgtcattgttttaca

SEQ ID NO: 42 - DNA, 68 bp, synthetic construct
ggggaccactttgtacaagaaagctgggtttcaatggtgatggtgatggtgccaacctgtacagcatg

SEQ ID NO: 43 - DNA, 62 bp, synthetic construct
ggggacaagtttgtacaaaaaagcaggcttcgccaccctgactatgatgagggtgctaaggc

SEQ ID NO: 44 - DNA, 73 bp, synthetic construct
ggggaccactttgtacaagaaagctgggtttcaatggtgatggtgatggtgaattgctgggaaagttctcagg

SEQ ID NO: 45 - DNA, 59 bp, synthetic construct
ggggacaagtttgtacaaaaaagcaggcttcgccacccatgatctttggcctgctaatc

SEQ ID NO: 46 - DNA, 69 bp, synthetic construct
ggggaccactttgtacaagaaagctgggtttcaatggtgatggtgatggtgaagggaaagtaataggag

SEQ ID NO: 47 - DNA, 59 bp, synthetic construct
ggggacaagtttgtacaaaaaagcaggcttcgccaccctatgggctttgttgttctatg

SEQ ID NO: 48 - DNA, 71 bp, synthetic construct
ggggaccactttgtacaagaaagctgggtttcaatggtgatggtgatggtggaaaaattctaacaaaattg

SEQ ID NO: 49 - DNA, 56 bp, synthetic construct
ggggacaagtttgtacaaaaaagcaggcttcgccaccatggttattccacatcttg

SEQ ID NO: 50 - DNA, 69 bp, synthetic construct
ggggaccactttgtacaagaaagctgggtttcaatggtgatggtgatggtgtctctgttgcagcaaacg

SEQ ID NO: 51 - DNA, 58 bp, synthetic construct
ggggacaagtttgtacaaaaaagcaggcttcgccaccatgttatttactttattattc

SEQ ID NO: 52 - DNA, 68 bp, synthetic construct
ggggaccactttgtacaagaaagctgggtttcaatggtgatggtgatggtgcagaaaaatcaaagagg

(19) United States
(12) Patent Application Publication
(10) Pub. No.: US 2006/0080744 A1
(43) Pub. Date: Apr. 13, 2006

(54) Novel chemokine-like polypeptides

(75) Inventors: Mark Ibberson, Gimel (CH); Christine Power, Thiory (FR); Achim Frauenschuh, Plan-les-Ouates (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Applied Research Systems ARS Holding N.V., Curacao (AN)

(21) Appl. No.: 10/530,104

(22) PCT Filed: Sep. 29, 2003
(86) PCT No.: PCT/EP03/50668

§ 371 Date: Aug. 2, 2005

(30) Foreign Application Priority Data
Oct. 4, 2002 (EP) 02022270.9

(51) Int. Cl. A01K 67/033; C07H 21/04; C12P 21/02; A61K 38/19; A61K 48/00; C07K 14/52

(52) U.S. Cl. 800/8; 424/85.1; 435/69.5; 435/320.1; 435/325; 514/44; 530/351; 530/388.23; 536/23.5

(57) ABSTRACT

The present invention discloses open reading frames (ORFs) in human genome encoding for novel chemokines-like polypeptides, and reagents related thereto including variants, mutants and fragments of said polypeptides, as well as ligands and antagonists directed against them. The invention provides methods for identifying and making these molecules, for preparing pharmaceutical compositions containing them, and for using them in the diagnosis, prevention and treatment of diseases.

### FIELD OF THE INVENTION

The present invention relates to nucleic acid sequences identified in human genome as encoding for novel polypeptides, more specifically for chemokine-like polypeptides.

### BACKGROUND OF THE INVENTION

The mammalian immune response is based on a series of complex, network-like interactions involving cellular components (such as lymphocytes or granulocytes) and soluble proteins, capable of modulating cellular activities (movement, proliferation, differentiation, etc.). Thus, there is considerable interest in the isolation and characterization of cell modulating factors, with the purpose of providing significant advancements in the diagnosis, prevention, and therapy of human disorders, in particular the ones associated to the immune system.

Chemokines are amongst these soluble proteins, since they are involved in the directional migration and activation of cells. This superfamily of small (70-130 amino acids), secreted, heparin-binding, pro-inflammatory proteins is known especially for the role in the extravasation of leukocytes from the blood to tissue localizations needing the recruitment of these cells (Baggiolini M et al., 1997; Yoshie O F et al., 2001; Fernandez E J and Lolis E, 2002).

Chemokines are not only functionally related but also structurally related, since they all contain a central region in which conserved Cysteines form intramolecular bonds. In particular, the number and the position of the most N-terminal of these conserved Cysteines in the mature polypeptides is the basic criteria for the generally recognized classification of chemokines, essentially divided between chemokines having a single or adjacent Cysteines (C—C chemokines), or chemokines having two Cysteines separated by 1-3 amino acids (C—X—C chemokines).

A series of membrane receptors, all heptahelical G-protein coupled receptors, are the binding partners that allow chemokines to exert their biological activity on the target cells. The physiological effects of chemokines result from a complex and integrated system of concurrent interactions. Different cells can present specific combinations of receptors according to their state and/or type. Moreover, chemokine receptors often have overlapping ligand specificity, so that a single receptor can bind different chemokines, as well a single chemokine can bind different receptors, still at high affinity.

Usually chemokines are produced at the site of an injury, inflammation, or other tissue alteration, and exert their activity in a paracrine or autocrine fashion. However, cell-type specific migration and activation in inflammatory and immune processes is not the sole activity of chemokines. Other physiological activities, such as hematopoiesis or angiogenesis, and pathological conditions, such as metastasis, transplant rejection, Alzehimer's disease or atherosclerosis, appear to be regulated by, at least, some of these proteins. In fact, chemokines and/or their receptors have been found considerably over-expressed and/or activated in several animal models or clinical samples (Haskell C A et al., 2002; Lucas A D and Greaves D R, 2001; Frederick M J and Clayman G L, 2001; Godessart N and Kunkel S L, 2001; Reape T J and Groot P H, 1999).

There are potential drawbacks in using chemokines as therapeutic agents (tendency to aggregate and promiscuous binding, in particular), but molecules having antagonistic properties against chemokines are widely considered as offering valuable opportunities for therapeutic intervention in disorders associated to excessive chemokine activities. The inhibition of specific chemokines and their receptors is considered a solution for preventing undesirable or uncontrolled cellular processes, such as recruitment or activation (Baggiolini M, 2001; Proudfoot A, 2000; Rossi D F and Zlotnik A, 2000).

The extensive sequencing programs and bioinformatics have made available a large amount of tools and information on human genome and physiology (Quinn-Senger K E et al., 2002; Browne M J, 2000). Such technologies were also used for discovering novel chemokines and receptors possibly providing new and useful therapeutic molecules and targets. Initially, chemokines genes were regularly mapped on chromosomes 4 and 17, in gene-rich areas of human genome (Nomiyama H et al., 2001), but the literature provides various approaches for characterizing novel chemokines by comparing the tissue-distribution of transcripts. Chemokines are usually expressed in lymphoid and other tissues but novel chemokines can have specific expression patterns and can be mapped to chromosomal loci different from the traditional gene clusters (WO 02/70706; Wells T N and Peitsch M C, 2000; Chantry D F et al., 1998; Rossi D et al., 1997).

Novel chemokines have been identified by applying strict homology criteria to known chemokines. However, since the actual content in polypeptide-encoding sequence in human genome for chemokines (as for any other protein family) is still unknown, the possibility still exists to identify DNA sequence encoding for polypeptides having chemotactic activities by applying alternative criteria in the analysis of Open Reading Frames (ORFs, that is, DNA sequences containing consecutive coding triplets of nucleotides, not interrupted by a termination codon and that can be potentially translated in a polypeptide) present in human genome.

### SUMMARY OF THE INVENTION

The invention is based upon the identification of Open Reading Frames (ORFs) in human genome encoding novel chemokine-like polypeptides.

Accordingly, the invention provides isolated polypeptides having the amino acid sequence given by SEQ ID NO: 2, 4, 6, 8, 10, 12, 14 or 16, and their mature forms, variants, and fragments, as polypeptides having chemotactic activity. The invention includes also the nucleic acids encoding them, vectors containing such nucleic acids, and cell containing these vectors or nucleic acids, as well as other related reagents such as fusion proteins, ligands, and antagonists.

The invention provides methods for identifying and making these molecules, for preparing pharmaceutical compositions containing them, and for using them in the diagnosis, prevention and treatment of diseases.

### DESCRIPTION OF THE FIGURES

FIG. 1: alignment of the ORF contained in the DNA sequence GNSQ_{—}1754 (SEQ ID NO: 1) with the protein sequence p1754 (SEQ ID NO: 2). The predicted N-terminal signal sequence is boxed. The predicted C-terminal alpha helix is underlined. The codons matching the original selection criteria are indicated with §. The arrows indicate the position of the primers CL_{—}1754_{—}5 (forward) and CL_{—}1754_{—}3 (reverse) in the ORF sequence.

FIG. 2: alignment of the ORF contained in the DNA sequence GNSQ_{—}0711 (SEQ ID NO: 3) with the protein sequence p0711 (SEQ ID NO: 4). The predicted N-terminal signal sequence is boxed. The predicted C-terminal alpha helix is underlined. The codons matching the original selection criteria are indicated with §. The arrows indicate the position of the primers CL_{—}0711_{—}5 (forward) and CL_{—}0711_{—}3 (reverse) in the ORF sequence.

FIG. 3: alignment of the ORF contained in the DNA sequence GNSQ_{—}2882 (SEQ ID NO: 5) with the protein sequence p2882 (SEQ ID NO: 6). The predicted N-terminal signal sequence is boxed. The predicted C-terminal alpha helix is underlined. The codons matching the original selection criteria are indicated with §.

FIG. 4: alignment of the ORF contained in the DNA sequence GNSQ_{—}4711 (SEQ ID NO: 7) with the protein sequence p4711 (SEQ ID NO: 8). The predicted N-terminal signal sequence is boxed. The predicted C-terminal alpha helix is underlined. The codons matching the original selection criteria are indicated with §.

FIG. 5: alignment of the ORF contained in the DNA sequence GNSQ_{—}4320 (SEQ ID NO: 9) with the protein sequence p4320 (SEQ ID NO: 10). The predicted N-terminal signal sequence is boxed. The predicted C-terminal alpha helix is underlined. The codons matching the original selection criteria are indicated with §. The arrows indicate the position of the primers CL_{—}4320_{—}5 (forward) and CL_{—}4320_{—}3 (reverse) in the ORF sequence.

FIG. 6: alignment of the ORF contained in the DNA sequence GNSQ_{—}5008 (SEQ ID NO: 11) with the protein sequence p5008 (SEQ ID NO: 12). The predicted N-terminal signal sequence is boxed. The predicted C-terminal alpha helix is underlined. The codons matching the original selection criteria are indicated with §. The arrows indicate the position of the primers CL_{—}5008_{—}5 (forward) and CL_{—}5008_{—}3 (reverse) in the ORF sequence.

FIG. 7: alignment of the ORF contained in the DNA sequence GNSQ_{—}0210 (SEQ ID NO: 13) with the protein sequence p0210 (SEQ ID NO: 14). The predicted N-terminal signal sequence is boxed. The predicted C-terminal alpha helix is underlined. The codons matching the original selection criteria are indicated with §. The arrows indicate the position of the primers CL_{—}0210_{—}5 (forward) and CL_{—}0210_{—}3 (reverse) in the ORF sequence.

FIG. 8: alignment of the ORF contained in the DNA sequence GNSQ_{—}4922 (SEQ ID NO: 15) with the protein sequence p4922 (SEQ ID NO: 16). The predicted N-terminal signal sequence is boxed. The predicted C-terminal alpha helix is underlined. The codons matching the original selection criteria are indicated with §. The arrows indicate the position of the primers CL_{—}4922_{—}5 (forward) and CL_{—}4922_{—}3 (reverse) in the ORF sequence.

FIG. 9: alignment of human CXCL chemokines with the CXC chemokine-like protein sequences of the invention p1754 (SEQ ID NO: 2), p0711 (SEQ ID NO: 4), p2882 (SEQ ID NO: 6), p0210 (SEQ ID NO: 14), and p4922 (SEQ ID NO: 16). The following human CXCL chemokines have been considered: CXCL1 (SWISSPROT Acc. No P09341), CXCL2 (SWISSPROT Acc. No P19875), CXCL3 (SWISSPROT Acc. No NP_{—}002081), CXCL4 (SWISSPROT Acc. No NP_{—}002610), CXCL5 (SWISSPROT Acc. No P42830), CXCL6 (SWISSPROT Acc. N° P80162), CXCL7 (SWISSPROT Acc. No P02775), CXCL8 (SWISSPROT Acc. No P10145), CXCL9 (SWISSPROT Acc. N° Q07325), CXCL10 (SWISSPROT Acc. N° P02778), CXCL11 (SWISSPROT Acc. No O14625). The protein sequences are divided according to the structure of the three main regions: the N-terminal region (containing the signal sequence), the central Cys-rich region (containing the conserved Cysteines matching the original selection criteria and indicated with §), and the C-terminal region (containing the predicted alpha helix).

FIG. 10: alignment of human CCL chemokines with the CXC chemokine-like protein sequences of the invention p4711 (SEQ ID NO: 8), p4320 (SEQ ID NO: 10), and GNSQ_{—}5008 (SEQ ID NO: 12). The following human CCL chemokines have been considered: CCL1 (SWISSPROT Acc. No P22362), CCL2 (SWISSPROT Acc. No P13500), CCL3 (SWISSPROT Acc. No P10147), CCL4 (SWISSPROT Acc. No P13236), CCL5 (SWISSPROT Acc. No P13501), CCL7 (SWISSPROT Acc. No P80098), CCL8 (SWISSPROT Acc. No P80075). The protein sequences are divided according to the structure of the three main regions: the N-terminal region (containing the signal sequence), the central Cys-rich region (containing the conserved Cysteines matching the original selection criteria and indicated with §), and the C-terminal region (containing the predicted alpha helix).

FIG. 11: Map of the pEAK12d expression vector.

### DETAILED DESCRIPTION OF THE INVENTION

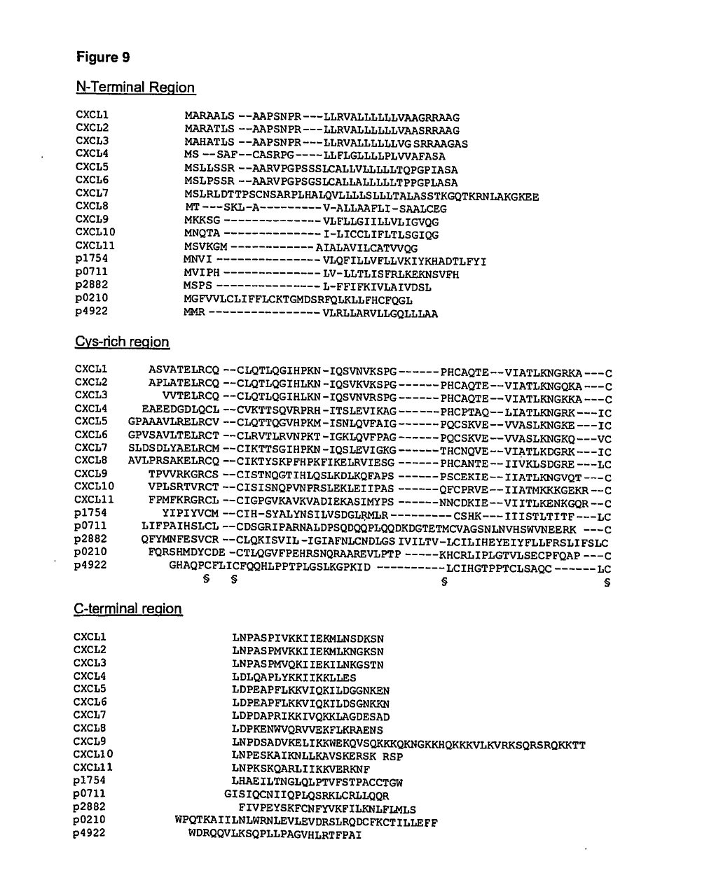

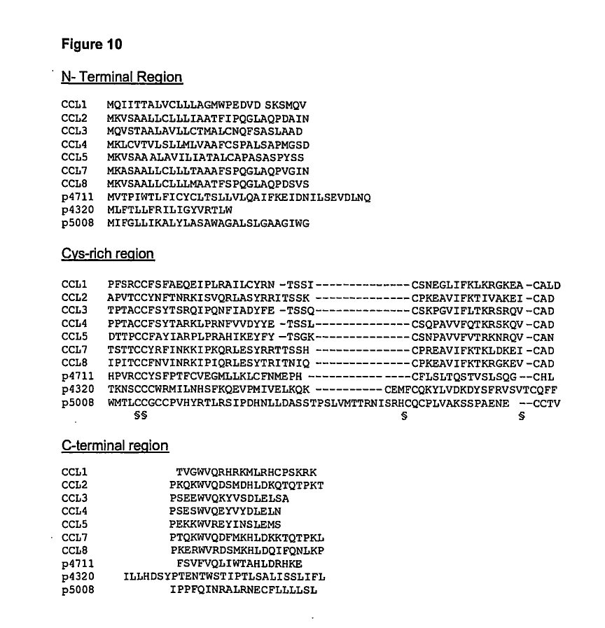

The main object of the present invention is to provide novel, isolated polypeptides having chemotactic activity selected from the group consisting of:
a) the amino acid sequences SEQ ID NO: 2, 4, 6, 8, 10, 12, 14 or 16;
b) the mature form of the polypeptides SEQ ID NO: 2, 4, 6, 8, 10, 12, 14 or 16;
c) the polypeptides comprising the Cysteine-rich region of SEQ ID NO: 2, 4, 6, 8, 10, 12, 14 or 16, as indicated in FIGS. 9 and 10;
d) the active variant of the amino acid sequence given by SEQ ID NO: 2, 4, 6, 8, 10, 12, 14 or 16 wherein any amino acid specified in the chosen sequence is non-conservatively substituted, provided that no more than 15% of the amino acid residues in the sequence are so changed;
e) the active fragments, precursors, salts, or derivatives of the amino acid sequences given in a) to d).

The novel polypeptides p1754 (SEQ ID NO: 2), p0711 (SEQ ID NO: 4), p2882 (SEQ ID NO: 6), p4711 (SEQ ID NO: 8), p4320 (SEQ ID NO: 10), p5008 ( SEQ ID NO: 12), p0210 (SEQ ID NO: 14), and p4922 (SEQ ID NO: 16) were identified on the basis of a consensus sequence for human chemokines in which the number and the position of selected amino acids (initial methionine, cysteines, and hydrophobic residues) are defined for protein sequence having length comparable to known chemokines.

The totality of amino acid sequences obtained by translating the known ORFs in the human genome were challenged using this consensus sequence, and the positive hits were further screened for the presence of predicted specific structural and functional “signatures” (a N-terminal signal sequence and a C-terminal alpha helix), and finally selected by comparing sequence features with known chemokines. Therefore, the novel polypeptides of the invention can be predicted to have chemotactic activities.

The terms “active” and “activity” refer to the chemotactic-like properties predicted for the chemokine-like amino acid sequences SEQ ID NO: 2, 4, 6, 8, 10, 12 14, or 16 in the present patent application.

Protein sequences having the indicated number of non-conservative substitutions can be identified using commonly available bioinformatic tools (Mulder N J and Apweiler R, 2002; Rehm B H, 2001).

In addition to such sequences, a series of polypeptides forms part of the disclosure of the invention. Being chemokines known to go through maturation processes including the proteolytic removal of N-terminal sequences (by signal peptidases and other proteolytic enzymes), the present patent application also claim the mature form of the polypeptides SEQ ID NO: 2, 4, 6, 8, 10, 12, 14 or 16. As mature form is intended any polypeptide showing chemotactic activity and resulting from in vivo (by the expressing cells or animals) or in vitro (by modifying the purified polypeptides with specific enzymes) post-translational maturation processes. Mature forms of chemokines resulting from C-terminal processing are also known (Ehlert J E et al., 1998). Other alternative mature forms can also result from the addition of chemical groups such as sugars or phosphates.

A further group of polypeptides of the invention are the polypeptides comprising the Cysteine-rich region of SEQ ID NO: 2, 4, 6, 8, 10, 12, 14 or 16, as indicated in FIG. 9 and 10, since the central Cysteine-rich region contains the essential structural and functional groups of chemokines.

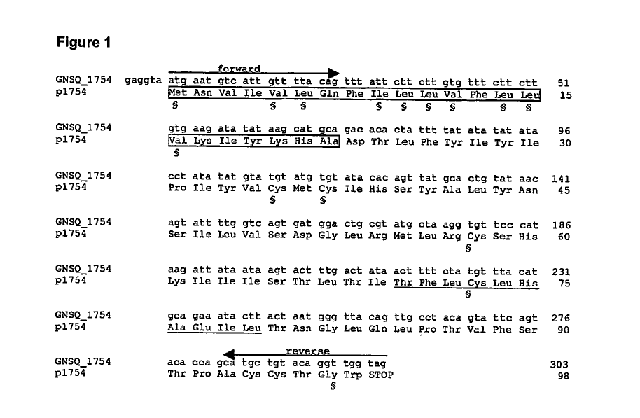

Other claimed polypeptides are the active variants of the amino acid sequences given by SEQ ID NO: 2, 4, 6, 8, 10, 12 14, or 16 wherein any amino acid specified in the chosen sequence is non-conservatively substituted, provided that no more than 15% of the amino acid residues in the sequence are so changed. The indicated percentage has to be measured over the novel amino acid sequences disclosed in FIGS. 1-8, and in particular over a segment of at least 40 amino acids containing the Cysteine-rich regions as indicated in FIGS. 9 and 10.

In accordance with the present invention, any substitution should be preferably a “conservative” or “safe” substitution, which is commonly defined a substitution introducing an amino acids having sufficiently similar chemical properties (e.g. a basic, positively charged amino acid should be replaced by another basic, positively charged amino acid), in order to preserve the structure and the biological function of the molecule.

The literature provide many models on which the selection of conservative amino acids substitutions can be performed on the basis of statistical and physico-chemical studies on the sequence and/or the structure of proteins (Rogov S I and Nekrasov A N, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The groups of synonymous amino acids and the groups of more preferred synonymous amino acids are shown in Table I.

Active variants having comparable, or even improved, activity with respect of corresponding chemokines may result from conventional mutagenesis technique of the encoding DNA, from combinatorial technologies at the level of encoding DNA sequence (such as DNA shuffling, phage display/selection), or from computer-aided design studies, followed by the validation for the desired activities as described in the prior art.

Specific, non-conservative mutations can be also introduced in the polypeptides of the invention with different purposes. Mutations reducing the affinity of the chemokine-like polypeptide for a receptor may increase its ability to be reused and recycled, potentially increasing its therapeutic potency (Robinson C R, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Further alternative polypeptides of the invention are active fragments, precursors, salt, or derivative of the amino acid sequences the above described sequences.

Fragments should present deletions of terminal or internal amino acids not altering their function, and should involve generally a few amino acids, e.g., under ten, and preferably under three, without removing or displacing amino acids which are critical to the functional conformation of the proteins.

The “precursors” are compounds which can be converted into the compounds of present invention by metabolic and enzymatic processing prior or after the administration to the cells or to the body.

The term “salts” herein refers to both salts of carboxyl groups and to acid addition salts of amino groups of the polypeptides of the present invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the invention or their analogs.

The term “derivatives” as herein used refers to derivatives which can be prepared from the functional groups present on the lateral chains of the amino acid moieties or on the amino- or carboxy-terminal groups according to known methods. Such molecules can result also from other modifications which do not normally alter primary sequence, for example in vivo or in vitro chemical derivativization of polypeptides (acetylation or carboxylation), those made by modifying the pattern of phosphorylation (introduction of phosphotyrosine, phosphoserine, or phosphothreonine residues) or glycosylation (by exposing the polypeptide to mammalian glycosylating enzymes) of a peptide during its synthesis and processing or in further processing steps. Alternatively, derivatives may include esters or aliphatic amides of the carboxyl-groups and N-acyl derivatives of free amino groups or O-acyl derivatives of free hydroxyl-groups and are formed with acyl-groups as for example alcanoyl- or aryl-groups.

The generation of the derivatives may involve a site-directed modification of an appropriate residue, in an internal or terminal position. The residues used for attachment should they have a side-chain amenable for polymer attachment (i.e., the side chain of an amino acid bearing a functional group, e.g. lysine, aspartic acid, glutamic acid, cysteine, histidine, etc.). Alternatively, a residue having a side chain amenable for polymer attachment can replace an amino acid of the polypeptide, or can be added in an internal or terminal position of the polypeptide. Also, the side chains of the genetically encoded amino acids can be chemically modified for polymer attachment, or unnatural amino acids with appropriate side chain functional groups can be employed. The preferred method of attachment employs a combination of peptide synthesis and chemical ligation. Advantageously, the attachment of a water-soluble polymer will be through a biodegradable linker, especially at the amino-terminal region of a protein. Such modification acts to provide the protein in a precursor (or “pro-drug”) form, that, upon degradation of the linker releases the protein without polymer modification.

Polymer attachment may be not only to the side chain of the amino acid naturally occurring in a specific position of the antagonist or to the side chain of a natural or unnatural amino acid that replaces the amino acid naturally occurring in a specific position of the antagonist, but also to a carbohydrate or other moiety that is attached to the side chain of the amino acid at the target position. Rare or unnatural amino acids can be also introduced by expressing the protein in specifically engineered bacterial strains (Bock A, 2001).

Variants of the polypeptides above indicated can be naturally occurring, being identified in organisms other than humans, or resulting from the translation of a single nucleotide polymorphism. Alternatively, artificial variants can be prepared by chemical synthesis, by site-directed mutagenesis techniques, or any other known technique suitable thereof, which provide a finite set of substantially corresponding mutated or shortened peptides or polypeptides which can be routinely obtained and tested by one of ordinary skill in the art using the teachings presented in the prior art.

The novel amino acid sequences disclosed in the present patent application can be used to provide different kind of reagents and molecules. Examples of these compounds are binding proteins or antibodies that can be identified using their full sequence or specific fragments, such as antigenic determinants. Peptide libraries can be used in known methods (Tribbick G, 2002) for screening and characterizing antibodies or other proteins binding the claimed amino acid sequences, and for identifying alternative forms of such polypeptides having similar binding properties.

The present patent application discloses also fusion proteins comprising any of the polypepudes described above. These polypeptides should contain protein sequence heterologous to the one disclosed In the present patent application, without significatively impairing the chemotactic activity and possibly providing additional properties. Examples of such properties are an easier purification procedure, a longer lasting half-life in body fluids, an additional binding moiety, the maturation by means of an endoproteolytic digestion, or extracellular localization. This latter feature is of particular importance for defining a specific group of fusion or chimeric proteins included in the above definition since it allows the claimed molecules to be localized in the space where not only isolation and purification of these polypeptides is facilitated, but also where generally chemokines and their receptor interact.

Design of the moieties, ligands, and linkers, as well methods and strategies for the construction, purification, detection and use of fusion proteins are disclosed in the literature (Nilsson J et al., 1997; Methods Enzymol, Vol. 326-328, Academic Press, 2000). The preferred protein sequences that can be comprised in the fusion proteins of the invention belong to these protein sequences: membrane-bound protein, immunoglobulin constant region, multimerization domains, extracellular proteins, signal peptide-containing proteins, export signal-containing proteins. Features of these sequences and their specific uses are disclosed in a detailed manner, for example, for albumin fusion proteins (WO 01/77137), fusion proteins including multimerization domain (WO 01/02440, WO 00/24782, WO 94/10308, WO 97/30161), immuno-conjugates (Garnett M C, 2001), or fusion protein including sequences allowing the purification of the recombinant products by affinity chromatography (Constans A, 2002; Burgess R R and Thompson N E, 2002; Lowe C R et al., 2001; Sheibani N, 1999).

Several studies on structure-activity features of chemokines indicate that these proteins bind and activate receptors by making use of the amino-terminal region. Proteolytic digestion, mutagenesis, or chemical modifications directed to amino acids in this region can generate compounds having antagonistic activity (Loetscher P and Clark-Lewis I, 2001; Lambeir A et al., 2001, Proost P et al., 2001). Thus, antagonistic molecules resulting from specific modifications (deletions, non-conservative substitutions, addition of chemical groups) of one or more residues in the amino-terminal region or in other regions of the corresponding chemokine are considered having therapeutic potential for inflammatory and autoimmune diseases (WO 02/28419; WO 00/27880; WO 99/33989; Schwarz M K and Wells T, 1999). Therefore, a further object of the present patent application is represented by such kind of antagonists generated by modifying the polypeptides of the invention.

The polypeptides of the invention can be used to generate and characterize ligands binding specifically to them. These molecules can be natural or artificial, very different from the chemical point of view (binding proteins, antibodies, molecularly imprinted polymers), and can be produced by applying the teachings in the art (WO 02/74938; Kuroiwa Y et al., 2002; Haupt K, 2002; van Dijk M A and van de Winkel J G, 2001; Gavilondo J V and Larrick J W, 2000). Such ligands can antagonize or inhibit the chemotactic activity of the polypeptide against which they have been generated. In particular, common and efficient ligands are represented by extracellular domain of a membrane-bound protein or antibodies, which can be in the form monoclonal, polyclonal, humanized antibody, or an antigen-binding fragment.

The polypeptides and the polypeptide-based derived reagents described above can be in alternative forms, according to the desired method of use and/or production, such as active conjugates or complexes with a molecule chosen amongst radioactive labels, fluorescent labels, biotin, or cytotoxic agents.

Specific molecules, such as peptide mimetics, can be also designed on the sequence and/or the structure of a polypeptde of the invention. Peptide mimetics (also called peptidomimetics) are peptides chemically modified at the level of amino acid side chains, of amino acid chirality, and/or of the peptide backbone. These alterations are intended to provide agonists or antagonists of the polypeptdes of the invention with improved preparation, potency and/or pharmaco kinetics features.

For example, when the peptide is susceptible to cleavage by peptidases following injection into the subject is a problem, replacement of a particularly sensitive peptide bond with a non-cleavable peptide mimetic can provide a peptide more stable and thus more useful as a therapeutic. Similarly, the replacement of an L-amino acid residue is a standard way of rendering the peptide less sensitive to proteolysis, and finally more similar to organic compounds other than peptides. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4-dinitrophenyl. Many other modifications providing increased potency, prolonged activity, easiness of purification, and/or increased half-life are disclosed in the prior art (WO 02/10195; Villain M et al., 2001).

Preferred alternative, synonymous groups for amino acids derivatives included in peptide mimetics are those defined in Table II. A non-exhaustive list of amino acid derivatives also include aminoisobutyric acid (Aib), hydroxyproline (Hyp), 1,2,3,4-tetrahydro-isoquinoline-3-COOH, indoline-2carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem AG, Switzerland; Bachem, USA).

Various methodologies for incorporating unnatural amino acids derivatives into proteins, using both in vitro and in vivo translation systems, to probe and/or improve protein structure and function are disclosed in the literature (Dougherty D A, 2000). Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design”, edited by Veerapandian P, Marcel Dekker Inc., pg. 557-663, 1997).

Another object of the present invention are isolated nucleic acids encoding for the polypeptides of the invention having chemotactic activity, the polypeptides binding to an antibody or a binding protein generated against them, the corresponding fusion proteins, or mutants having antagonistic activity as disclosed above. Preferably, these nucleic acids should comprise a DNA sequence selected from the group consisting of SEQ ID NO: 1, 3, 5, 7, 9, 11, 13, or 15, or the complement of said DNA sequences.

Alternatively, the nucleic acids of the invention should hybridize under high stringency conditions, or exhibits at least about 85% identity over a stretch of at least about 30 nucleotides, with a nucleic acid selected from the group consisting of SEQ ID NO: 1, 3, 5, 7, 9, 11, 13, or 15 or a complement of said DNA sequences. A further object of the present invention is therefore the polypeptides encoded by these purified nucleic acids.

The wording “high stringency conditions” refers to conditions in a hybridization reaction that facilitate the association of very similar molecules and consist in the overnight incubation at 60°-65° C. in a solution comprising 50% formamide, 5×SSC (150 m M NaCl, 15 m M trisodium citrate), 50 mM sodium phosphate (pH 76), 5× Denhardt's solution, 10% dextran sulphate, and 20 microgram/ml denatured, sheared salmon sperm DNA, followed by washing the filters in 0.1×SSC at the same temperature.

These nucleic acids, including nucleotide sequences substantially the same, can be comprised in plasmids, vectors and any other DNA construct which can be used for maintaining, modifying, introducing, or expressing the encoded polypeptide. In particular, vectors wherein said nucleic acid molecule is operatively linked to expression control sequences can allow expression in prokaryotic or eukaryotic host cells of the encoded polypeptide.

The wording “nucleotide sequences substantially the same” includes all other nucleic acid sequences that, by virtue of the degeneracy of the genetic code, also code for the given amino acid sequences. In this sense, the literature provides indications on preferred or optimized codons for recombinant expression (Kane J F et al., 1995).

The nucleic acids and the vectors can be introduced into cells with different purposes, generating transgenic cells and organisms. A process for producing cells capable of expressing a polypeptide of the invention comprises genetically engineering cells with such vectors and nucleic acids.

In particular, host cells (e.g. bacterial cells) can be modified by transformation for allowing the transient or stable expression of the polypeptides encoded by the nucleic acids and the vectors of the invention. Alternatively, said molecules can be used to generate transgenic animal cells or non-human animals (by non-/homologous recombination or by any other method allowing their stable integration and maintenance), having a constitutive or inducible altered expression levels (i.e. enhanced or reduced) of the polypeptides of the invention, when the level is compared with the normal expression levels. Such precise modifications can be obtained by making use of the nucleic acids of the inventions and of technologies associated, for example, to gene therapy (Meth. Enzymol., vol. 346, 2002) or to site-specific recombinases (Kolb A F, 2002). Model systems based on the chemokine-like polypeptides disclosed in the present patent application for the systematic study of their function can be also generated by gene targeting into human cell lines (Bunz F, 2002).

The polypeptides of the invention can be prepared by any method known in the art, including recombinant DNA-related technologies, and chemical synthesis technologies. In particular, a method for making a polypeptide of the invention may comprise culturing a host or transgenic cell as described above under conditions in which the nucleic acid or vector is expressed, and recovering the polypeptide encoded by said nucleic acid or vector from the culture. For example, when the vector expresses the polypeptide as a fusion protein with an extracellular or signal-peptide containing proteins, the recombinant product can be secreted in the extracellular space, and can be more easily collected and purified from cultured cells in view of further processing or, alternatively, the cells can be directly used or administered.

The DNA sequence coding for the proteins of the invention can be inserted and ligated into a suitable episomal or non-/homologously integrating vectors, which can be introduced in the appropriate host cells by any suitable means (transformation, transfection, conjugation, protoplast fusion, electroporation, calcium phosphate-precipitation, direct microinjection, etc.). Factors of importance in selecting a particular plasmid or viral vector include: the ease with which recipient cells that contain the vector, may be recognized and selected from those recipient cells which do not contain the vector; the number of copies of the vector which are desired in a particular host; and whether it is desirable to be able to “shuttle” the vector between host cells of different species.

The vectors should allow the expression of the isolated or fusion protein including the polypeptide of the invention by prokaryotic or eukaryotic host cells under the control of transcriptional initiation/termination regulatory sequences, which are chosen to be constitutively active or inducible in said cell. A cell line substantially enriched in such cells can be then isolated to provide a stable cell line.

Different transcriptional and translational regulatory sequences may be employed for eukaryotic hosts (e.g. yeasts, insect, plant, or mammalian cells), depending on the nature of the host. They may be derived form viral sources, such as adenovirus, bovine papilloma virus, Simian virus or the like, where the regulatory signals are associated with a particular gene which has a high level of expression. Examples are the TK promoter of the Herpes virus, the SV40 early promoter, the yeast GAL4 gene promoter, etc. Transcriptional initiation regulatory signals may be selected which allow for repression and activation, so that expression of the genes can be modulated. The cells stably transformed by the introduced DNA can be selected by introducing one or more markers allowing the selection of host cells that contain the expression vector. The marker may also provide for phototrophy to an auxotropic host, biocide resistance, e.g. antibiotics, or heavy metals such as copper, or the like. The selectable marker gene can either be directly linked to the DNA gene sequences to be expressed, or introduced into the same cell by co-transfection.

Host cells may be either prokaryotic or eukaryotic. Preferred are eukaryotic hosts, e.g. mammalian cells, such as human, monkey, mouse, and Chinese Hamster Ovary (CHO) cells, because they provide post-translational modifications to proteins, including correct folding and glycosylation. Also yeast cells can carry out post-translational peptide modifications including glycosylation. A number of recombinant DNA strategies exist which utilize strong promoter sequences and high copy number of plasmids that can be utilized for production of the desired proteins in yeast. Yeast recognizes leader sequences in cloned mammalian gene products and secretes peptides bearing leader sequences (i.e., pre-peptides).

The above mentioned embodiments of the invention can be achieved by combining the disclosure provided by the present patent application on the sequence of novel chemokine-like polypeptides with the knowledge of common molecular biology techniques.

Many books and reviews provides teachings on how to clone and produce recombinant proteins using vectors and prokaryotic or eukaryotic host cells, such as some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

Moreover, updated and more focused literature provides an overview of the technologies for expressing polypeptides in a high-throughput manner (Chambers S P, 2002; Coleman T A, et al., 1997), of the cell systems and the processes used industrially for the large-scale production of recombinant proteins having therapeutic applications (Andersen D C and Krummen L, 2002; Chu L and Robinson D K, 2001), and of alternative eukaryotic expression systems for expressing the polypeptide of interest, which may have considerable potential for the economic production of the desired protein, such the ones based on transgenic plants (Giddings G, 2001) or the yeast Pichia pastoris (Lin Cereghino G P et al., 2002). Recombinant protein products can be rapidly monitored with various analytical technologies during purification to verify the amount and the quantity of the expressed polypeptides (Baker K N et al., 2002), as well as to check if there Is problem of bioequivalence and immunogenicity (Schellekens H, 2002; Gendel S M, 2002).

Totally synthetic chemokines are disclosed in the literature (Brown A et al., 1996), and many examples of chemical synthesis technologies, which can be effectively applied for the chemokine-like polypeptides of the invention given their short length, are available in the literature, as solid phase or liquid phase synthesis technologies. for example, the amino acid corresponding to the carboxy-terminus of the peptide to be synthetized is bound to a support which is insoluble in organic solvents, and by alternate repetition of reactions, one wherein amino acids with their amino groups and side chain functional groups protected with appropriate protective groups are condensed one by one in order from the carboxy-terminus to the amino-terminus, and one where the amino acids bound to the resin or the protective group of the amino groups of the peptides are released, the peptide chain is thus extended in this manner.

Solid phase synthesis methods are largely classified by the tBoc method and the Fmoc method, depending on the type of protective group used. Typically used protective groups include tBoc (t-butoxycarbonyl), Cl-Z (2-chlorobenzyloxycarbonyl), Br-Z (2-bromobenzyloxycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Cl2-Bzl (2,6-dichlorobenzyl) for the amino groups; NO2 (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or trifluoromethane sulfonic acid for the Boc method, and with TFA for the Fmoc method.

The purification of the polypeptides of the invention can be carried out by any one of the methods known for this purpose, i.e. any conventional procedure involving extraction, precipitation, chromatography, electrophoresis, or the like. A further purification procedure that may be used in preference for purifying the protein of the invention is affinity chromatography using monoclonal antibodies or affinity groups, which bind the target protein and which are produced and immobilized on a gel matrix contained within a column. Impure preparations containing the proteins are passed through the column. The protein will be bound to the column by heparin or by the specific antibody while the impurities will pass through. After washing, the protein is eluted from the gel by a change in pH or ionic strength. Alternatively, HPLC (High Performance Liquid Chromatography) can be used. The elution can be carried using a water-acetonitrile-based solvent commonly employed for protein purification.

The disclosure of the novel polypeptides of the invention, and the reagents disclosed in connection to them (antibodies, nucleic acids, cells) allows also to screen and characterize compounds that enhance or reduce their expression level into a cell or in an animal. Examples of compounds that can reduce or block the expression of the chemokine-like polypeptides are antisense oligonucleotides (Stein C A, 2001) or small interfering, double stranded RNA molecules that can trigger RNA interference-mediated silencing (Paddison P J et al., 2002; Lewis D L et al., 2002). These compounds are intended as antagonists (in addition to the ones above described in connection to mutants and ligands) in the context of the possible mechanism of antagonism for blocking cytokine/chemokine-controlled pathways as defined in the literature (Choy E H and Panayi G S, 2001; Dower S K, 2000).

“Oligonucleotides” refers to either a single stranded polydeoxynucleotide or two complementary polydeoxynucleotide strands that may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The invention includes purified preparations of the compounds of the invention (polypeptides, nucleic acids, cells, etc.). Purified preparations, as used herein, refers to the preparations containing at least 1%, preferably at least 5%, by dry weight of the compound of the invention.

The present patent application discloses a series of novel chemokine-like polypeptides and of related reagents having several possible applications. In particular, whenever an increase in the chemotactic activity of a polypeptide of the invention is desirable in the therapy or in the prevention of a disease, reagents such as the disclosed chemokine-like polypeptides, the corresponding fusion proteins and peptide mimetics, the encoding nucleic acids, the expressing cells, or the compounds enhancing their expression can be used.

Therefore, the present invention discloses pharmaceutical compositions for the treatment or prevention of diseases needing an increase in the chemotactic activity of a polypeptide of the invention, which contain one of the disclosed chemokine-like polypeptides, the corresponding fusion proteins and peptide mimetics, the encoding nucleic acids, the expressing cells, or the compounds enhancing their expression, as active ingredient.

The process for the preparation of these pharmaceutical compositions comprises combining the disclosed chemokine-like polypeptides, the corresponding fusion proteins and peptide mimetics, the encoding nucleic acids, the expressing cells, or the compounds enhancing their expression, together with a pharmaceutically acceptable carrier.

Methods for the treatment or prevention of diseases needing an increase in the chemotactic activity of a polypeptide of the invention, comprise the administration of a therapeutically effective amount of the disclosed chemokine-like polypeptides, the corresponding fusion proteins and peptide mimetics, the encoding nucleic acids, the expressing cells, or the compounds enhancing their expression.

Amongst the reagents disclosed in the present patent application, the ligands, the antagonists or the compounds reducing the expression or the activity of polypeptides of the invention have several applications, and in particular they can be used in the therapy or in the diagnosis of a disease associated to the excessive chemotactic activity of a polypeptide of the invention.

Therefore, the present invention discloses pharmaceutical compositions for the treatment or prevention of diseases associated to the excessive chemotactic activity of a polypeptide of the invention, which contain one of the ligands, antagonists, or compounds reducing the expression or the activity of such poly peptides, as active ingredient.

The process for the preparation of these pharmaceutical compositions comprises combining the ligand, the antagonist, or the compound, together with a pharmaceutically acceptable carrier.

Methods for the treatment or prevention of diseases associated to the excessive chemotactic activity of the polypeptide of the invention comprise the administration of a therapeutically effective amount of the antagonist, the ligand or of the compound.

The pharmaceutical compositions of the invention may contain, in addition to chemokine-like polypeptide or to the related reagent, suitable pharmaceutically acceptable carriers, biologically compatible vehicles and additives which are suitable for administration to an animal (for example, physiological saline) and eventually comprising auxiliaries (like excipients, stabilizers, adjuvants, or diluents) which facilitate the processing of the active compound into preparations which can be used pharmaceutically.

The pharmaceutical compositions may be formulated in any acceptable way to meet the needs of the mode of administration. For example, of biomaterials, sugar-macromolecule conjugates, hydrogels, polyethylene glycol and other natural or synthetic polymers can be used for improving the active ingredients in terms of drug delivery efficacy. Technologies and models to validate a specific mode of administration are disclosed in literature (Davis B G and Robinson M A, 2002; Gupta P et al., 2002; Luo B and Prestwich G D, 2001; Cleland J L et al., 2001; Pillai O and Panchagnula R, 2001).

Polymers suitable for these purposes are biocompatible, namely, they are non-toxic to biological systems, and many such polymers are known. Such polymers may be hydrophobic or hydrophilic in nature, biodegradable, non-biodegradable, or a combination thereof. These polymers include natural polymers (such as collagen, gelatin, cellulose, hyaluronic acid), as well as synthetic polymers (such as polyesters, polyorthoesters, polyanhydrides). Examples of hydrophobic non-degradable polymers include polydimethyl siloxanes, polyurethanes, polytetrafluoroethylenes, polyethylenes, polyvinyl chlorides, and polymethyl methaerylates. Examples of hydrophilic non-degradable polymers include poly(2-hydroxyethyl methacrylate), polyvinyl alcohol, poly(N-vinyl pyrrolidone), polyalkylenes, polyacrylamide, and copolymers thereof. Preferred polymers comprise as a sequential repeat unit ethylene oxide, such as polyethylene glycol (PEG).

Any accepted mode of administration can be used and determined by those skilled in the art to establish the desired blood levels of the active ingredients. For example, administration may be by various parenteral routes such as subcutaneous, intravenous, intradermal, intramuscular, intraperitoneal, intranasal, transdermal, oral, or buccal routes. The pharmaceutical compositions of the present invention can also be administered in sustained or controlled release dosage forms, including depot injections, osmotic pumps, and the like, for the prolonged administration of the polypeptide at a predetermined rate, preferably in unit dosage forms suitable for single administration of precise dosages.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl oleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the excipient.

The wording “therapeutically effective amount” refers to an amount of the active ingredients that is sufficient to affect the course and the severity of the disease, leading to the reduction or remission of such pathology. The effective amount will depend on the route of administration and the condition of the patient.

The wording “pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution. Carriers can be selected also from starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the various oils, including those of petroleum, animal, vegetable or synthetic origin (peanut oil, soybean oil, mineral oil, sesame oil).

It is understood that the dosage administered will be dependent upon the age, sex, health, and weight of the recipient, kind of concurrent treatment, if any, frequency of treatment, and the nature of the effect desired. The dosage will be tailored to the individual subject, as is understood and determinable by one of skill in the art. The total dose required for each treatment may be administered by multiple doses or in a single dose. The pharmaceutical composition of the present invention may be administered alone or in conjunction with other therapeutics directed to the condition, or directed to other symptoms of the condition. Usually a daily dosage of active ingredient is comprised between 0.01 to 100 milligrams per kilogram of body weight per day. Ordinarily 1 to 40 milligrams per kilogram per day given in divided doses or in sustained release form is effective to obtain the desired results. Second or subsequent administrations can be performed at a dosage, which is the same, less than, or greater than the initial or previous dose administered to the individual.

Apart from the methods having a therapeutic or a production purpose, several other methods can make use of the chemokine-like polypeptides and of the related reagents disclosed in the present patent application.

In a first example, a method for screening candidate compounds effective to treat a disease related to a chemokine-like polypeptides of the invention, comprises:
(a) contacting host cells expressing such polypeptide, transgenic non-human animals, or transgenic animal cells having enhanced or reduced expression levels of the polypeptide, with a candidate compound; and
(b) determining the effect of the compound on the animal or on the cell.

In a second example, a method for identifying a candidate compound as an antagonist/inhibitor or agonist/activator of a polypeptide of the invention comprises:
(a) contacting the polypeptide, the compound, and a mammalian cell or a mammallian cell membrane; and
(b) measuring whether the molecule blocks or enhances the interaction of the polypeptide, or the response that results from such interaction, with the mammalian cell or the mammalian cell membrane.

In a third example, methods for determining the activity and/or the presence of the polypeptide of the invention in a sample, can detect either the polypeptide or the encoding RNA/DNA. Thus, such a method comprises:
(a) providing a protein-containing sample;
(b) contacting said sample with a ligand of the invention; and
(c) determining the presence of said ligand bound to said polypeptide, thereby determining the activity and/or the presence of polypeptide in said sample.

Alternatively, the method comprises:
(a) providing a nucleic acids-containing sample;
(b) contacting said sample with a nucleic acid of the invention; and
(c) determining the hybridizauon of said nucleic acid with a nucleic acid into the sample, thereby determining the presence of the nucleic acid in the sample.

In this context, primer sequences containing the sequences SEQ ID NO: 17-28 (Table III) can be used as well for determining the presence or the amount of a transcript or of a nucleic acid encoding a polypeptide of invention in a sample by means of Polymerase Chain Reaction amplification.

A further object of the present invention are kits for measuring the activity and/or the presence of chemokine-like polypeptide of the invention in a sample comprising one or more of the reagents disclosed in the present patent application: a chemokine-like polypeptide of the invention, an antagonist, ligand or peptide mimetic, an isolated nucleic acid or the vector, a pharmaceutical composition, an expressing cell, a compound increasing or decreasing the expression levels, and/or primer sequences containing the sequences SEQ ID NO: 17-28.

Those kits can be used for in vitro diagnostic or screenings methods, and their actual composition should be adapted to the specific format of the sample (e.g. biological sample tissue from a patient), and the molecular species to be measured. For example, if it is desired to measure the concentration of the chemokine-like polypeptide, the kit may contain an antibody and the corresponding protein in a purified form to compare the signal obtained in Western blot. Alternatively, if it is desired to measure the concentration of the transcript for the chemokine-like polypeptide, the kit may contain a specific nucleic acid probe designed on the corresponding ORF sequence, or may be in the form of nucleic acid array containing such probe, or the primer sequences disclosed as SEQ ID NO: 17-28 (Table III). The kits can be also in the form of protein-, peptide mimetic-, or cell-based microarrays (Templin M F et al., 2002; Pellois J P et al., 2002; Blagoev B and Pandey A, 2001), allowing high-throughput proteomics studies, by making use of the proteins, peptide mimetics and cells disclosed in the present patent application.

The novel chemokine-like polypeptides of the invention and the related reagents that may be useful, as active ingredients in pharmaceutical compositions appropriately formulated, in the treatment or prevention of diseases such as cell proliferative disorders, autoimmune/inflammatory disorders, cardiovascular disorders, neurological disorders, developmental disorders, metabolic disorder, infections and other pathological conditions. In particular, given the known properties of chemokines, the disclosed polypeptides and reagents should address conditions involving abnormal or defective cell migration. Non-limitative examples of such conditions are the following: arthritis, rheumatoid arthritis (RA), psoriatic arthritis, osteoarthritis, systemic lupus erythematosus (SLE), systemic sclerosis, scleroderma, polymyositis, glomerulonephritis, fibrosis, lung fibrosis and inflammation, allergic or hypersensitvity diseases, dermatitis, asthma, chronic obstructive pulmonary disease (COPD), inflammatory bowel disease (IBD), Crohn's diseases, ulcerative colitis, multiple sclerosis, septic shock, HIV infection, transplant rejection, wound healing, metastasis, endometriosis, hepatitis, liver fibrosis, cancer, analgesia, and vascular inflammation related to atherosclerosis.

The therapeutic applications of the polypeptides of the invention and of the related reagents can be evaluated (in terms or safety, pharmacokinetics and efficacy) by the means of the in vivo or in vitro assays making use of animal cell, tissues and models (Coleman R A et al., 2001; Li A P, 2001; Methods Mol. Biol vol. 138, “Chemokines Protocols”, edited by Proudfoot A et al., Humana Press Inc., 2000; Methods Enzymol, vol. 287 and 288, Academic Press, 1997), or by the means of in silico, computational approaches (Johnson D E and Wolfgang G H, 2000), known for the validation of chemokines and other biological products during drug discovery and preclinical development.

All publications, patents and patent applications cited herein are incorporated in full by reference for all purposes.

The invention will now be described with reference to the specific embodiments by means of the following Examples, which should not be construed as in any way limiting the present invention. The content of the description comprises all modifications and substitutions which can be practiced by a person skilled in the art in light of the above teachings and, therefore, without extending beyond the meaning and purpose of the claims.

### EXAMPLES

### Example 1

### Selection of Chemokine-Like Open Reading Frames (ORFS) from Human Genome

Perl (Practical Extraction and Report Language) is a programming language having powerful pattern matching functions into large text data files allowing the extraction of information from genomic DNA sequences, starting from an alpha-numerical expression describing a defined consensus sequence (Stein L D, 2001).

A Perl script was used to retrieve novel open reading frames (ORFs), having chemokine-like features, in a FASTA-formatted sequence file containing the NCBI genome (build 28). After translating the genomic DNA sequence into the six possible reading frames (3 forward and 3 reverse), each of these translated sequences was then tested for a match against a pattern designed to detect to chemokine-like proteins, which was elaborated comparing multiple sequence alignments of known chemokines. The following pattern, fitting all the aligned sequences, was adopted:
{M}-{X}₃₋₁₂-{L or I or V}₁₋₃—{X}₀₋₂-{L or I or V}₂₋₄—{X}₀₋₂-{L or I or V}₁₋₃—{X}₁₀₋₃₀—{C}—(X)₁₀₋₃₀—C—(X)₂₀₋₄₀—C—{X}₁₂₋₂₀—{C}—{X}₁₅₋₄₀ STOP

The letter(s) between brackets represented alternative amino acids (in one-letter code) which should be present the number of times indicated in subscript characters. This expression, which describes the entire family of sequences on the basis of the respective positioning of the initial methionine, hydrophobic residues, and conserved cysteines on the linear sequence, can be transformed in Perl language as follows:
M[ˆ\*]{3,12}[LIV]{1,3}[ˆ\*]{0,2}[LIV]{2,4}[ˆ\*]{0,2}[LIV]{1,3}[ˆ\*]{10,30}C[ˆ\*]{0,3}C[ˆ\*]{20,40}C[ˆ\*]{12,20}C[ˆ\*]{15,40}[\*]

A total of FASTA-formatted 7974 ORFs matching the pattern were compared to known proteins present in protein databases (SwissProt/Trembl and Derwent GENESEQ) using a rapid searching program for local alignments between a query and a hit sequence based on Basic Local Alignment Search Tool (BLAST, BLASTX) and ClustalW algorithms (Altschul S F et al., 1990; Pearson W R and Miller W, 1992; Gish W and States D J, 1993). BLAST parameters used were: Comparison matrix=BLOSUM62; word length=3; .E value cutoff=10; Gap opening and extension=default; No filter.

The sequences obtained from this first screening were further selected using additional criteria. 2441 ORFs showing at least 70% of homology with known proteins in protein databases were eliminated. The remaining 5533 ORFs were filtered using 2 neural network-based algorithms developed for the prediction (probability at least 0.7) of a N-terminal signal peptide and of an alpha helix secondary structure having at least 5 amino acids within the C-terminal 30 amino acids (a hallmark of the IL8-like fold) with high confidence. The resulting 253 ORFs, which were predicted as containing these features, were transformed in text format and were compared to known chemokines, searching manually for the best alignments. This further refinement, based on the qualitative assessment of the alignments, led to the selection of eight chemokine-like encoding ORFs for which all criteria for the prediction (sequence length, cysteine spacing, N-terminal signal sequence, C-terminal alpha helix) were fulfilled, making them comparable to known chemokines.

The DNA sequence GNSQ_{—}1754 (SEQ ID NO: 1), belonging to human chromosome 13, contains an ORF encoding for the 98-amino acid long protein sequence p1754 (SEQ ID NO: 2), which, according to the prediction, presents a 22-amino acid long signal sequence and an alpha helix covering the residues 70-79 (FIG. 1).

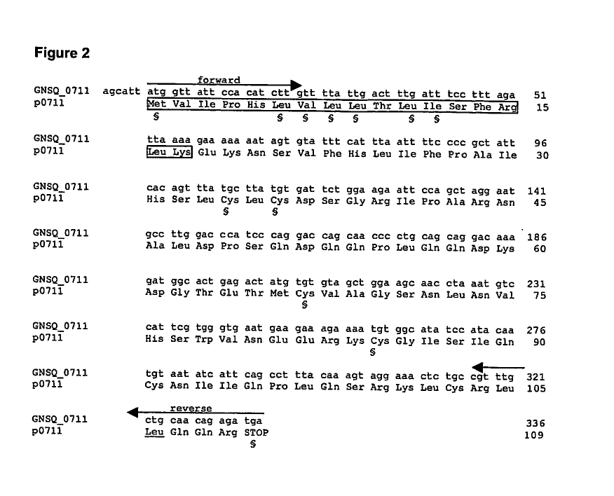

The DNA sequence GNSQ_{—}0711 (SEQ ID NO: 3), belonging to human chromosome 16, contains an ORF encoding for the 109-amino acid long protein sequence p0711 (SEQ ID NO: 4), which, according to the prediction, presents a 17-amino acid long signal sequence and an alpha helix covering the residues 98-106 (FIG. 2).

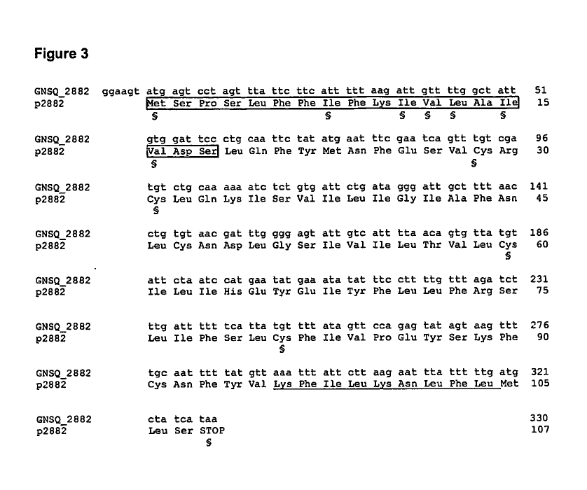

The DNA sequence GNSQ_{—}2882 (SEQ ID NO: 5), belonging to human chromosome 6, contains an ORF encoding for the 107-amino acid long protein sequence p2882 (SEQ ID NO: 6), which, according to the prediction, presents a 18-amino acid long signal sequence and an alpha helix covering the residues 96-104 (FIG. 3).

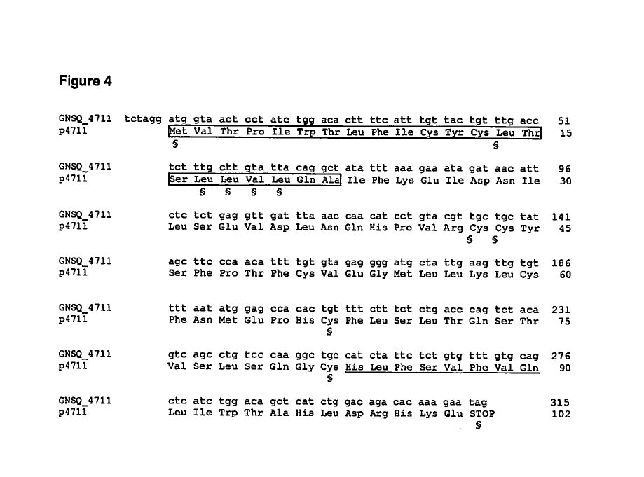

The DNA sequence GNSQ_{—}4711 (SEQ ID NO: 7), belonging to human chromosome 3, contains an ORF encoding for the 102-amino acid long protein sequence p4711 (SEQ ID NO: 8), which, according to the prediction, presents a 22-amino acid long signal sequence and an alpha helix covering the residues 83-97 (FIG. 4).

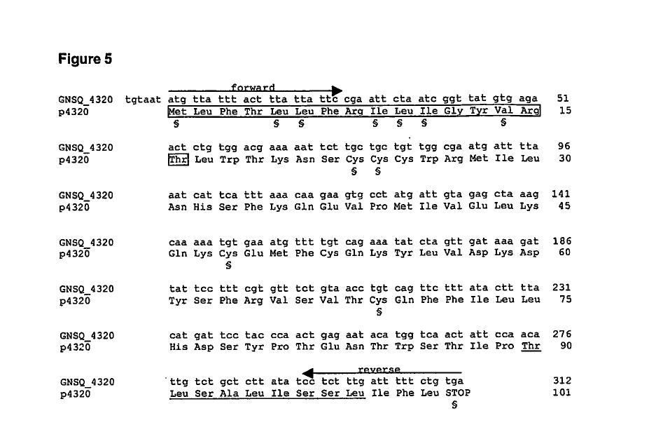

The DNA sequence GNSQ_{—}4320 (SEQ ID NO: 9), belonging to human chromosome 3, contains an ORF encoding for the 101-amino acid long protein sequence p4320 (SEQ ID NO: 10), which, according to the prediction, presents a 16-amino acid long signal sequence and an alpha helix covering the residues 90-98 (FIG. 5).

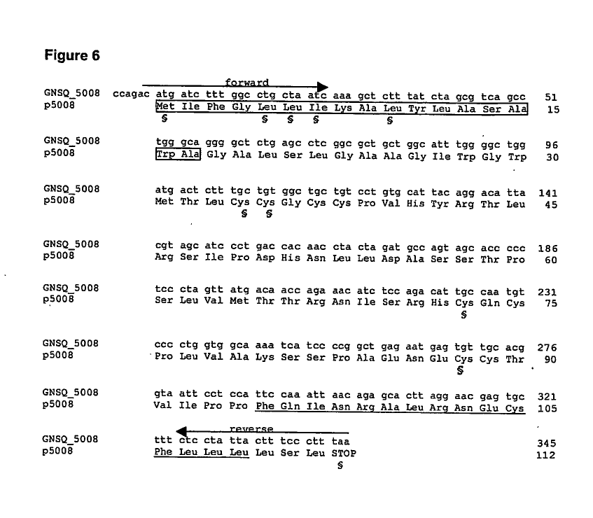

The DNA sequence GNSQ_{—}5008 (SEQ ID NO: 11), belonging to human chromosome 12, contains an ORF encoding for the 112-amino acid long protein sequence p5008 (SEQ ID NO: 12), which, according to the prediction, presents a 17-amino acid long signal sequence and an alpha helix covering the residues 95-109 (FIG. 6).

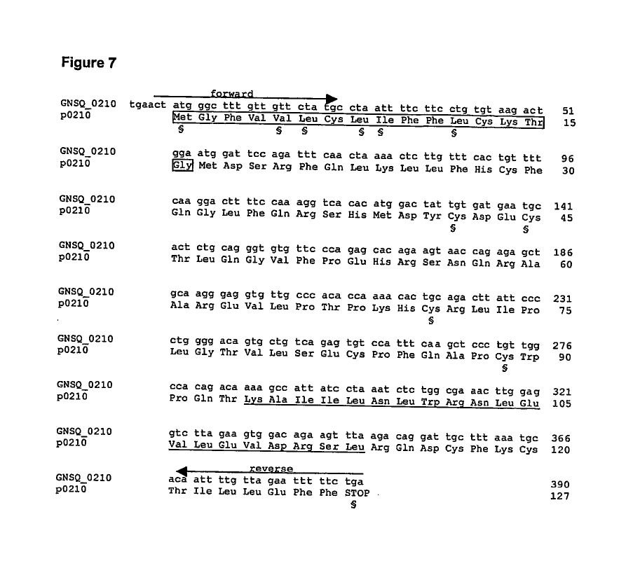

The DNA sequence GNSQ_{—}0210 (SEQ ID NO: 13), belonging to human chromosome 7, contains an ORF encoding for the 127-amino acid long protein sequence p0210 (SEQ ID NO: 14), which, according to the prediction, presents a 16-amino acid long signal sequence and an alpha helix covering the residues 94-113 (FIG. 7).

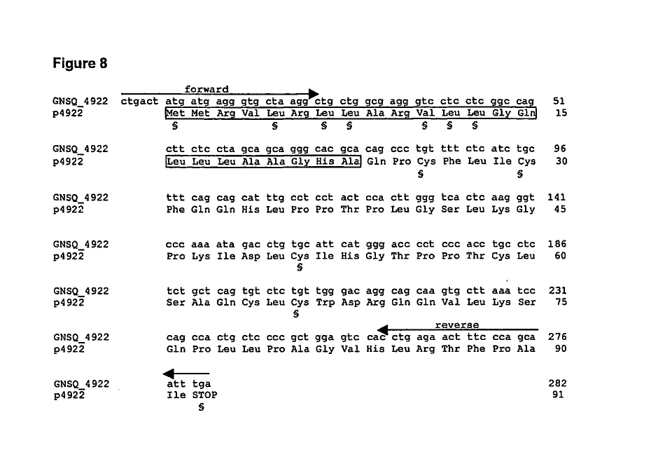

The DNA sequence GNSQ_{—}4922 (SEQ ID NO: 15), belonging to human chromosome 10, contains an ORF encoding for the 91-amino acid long protein sequence p4922 (SEQ ID NO: 14), which, according to the prediction, presents a 23-amino acid long signal sequence and an alpha helix covering the residues 67-74 (FIG. 8).

Amongst these sequences characterized as encoding chemokine-like polypeptides, five of them (p1754, p0711, p2882, p0210, and p4922) present a central Cys-rich region in which the first two Cysteines are separated by 1-3 amino acids, and can be compared with known C—X—C chemokines (FIG. 9). The remaining three sequences (p4711, p4320, and p5008) present two adjacent Cysteines at the beginning of such region, and therefore can be compared with known C—C chemokines (FIG. 10).

### Example 2

### Cloning of the Novel Chemokine-Like ORFs from Human Genomic DNA

Six of the eight above-defined chemokine-like ORFs (GNSQ_{—}1754, GNSQ_{—}4922, GNSQ_{—}5008, GNSQ_{—}0210, GNSQ_{—}0711, and GNSQ_{—}4320) were first cloned from human genomic DNA into a cloning vector, and then transferred into an expression vector using Polymerase Chain Reaction (PCR), with pairs of forward/reverse primers specific for each ORF (see arrows in FIGS. 1, 2, and 5-8).

The cloning primers (CL series; Table III), having a length comprised between 19 and 25 bases, were designed for amplifying each ORF, using human genomic DNA as template. The forward primers start from the initial ATG or a few nucleotides before. The reverse primers are complementary to the 3′ end of the ORF, including the stop codon.

The PCR was performed by mixing the following components in each ORF-specific reaction (total volume of 50 μl in double-distilled water):
150 ng human genomic DNA (Clontech)
1.2 μM primers (0.6 μM each primer)
240 μM dNTP (Invitrogen)
0.5 μl AmpliTaq (2.5 Units; Applied Biosystems)
5 μl AmpliTaq buffer 10× (Applied Biosystems)

The PCR reactions were performed using an initial denaturing step if 94° C. for 2 minutes, followed by 30 cycles:
94° C. for 30 seconds
55° C. for 30 seconds
72° C. for 30 seconds

After a final elongation step of 72° C. for 10 minutes, the PCR products were directly subcloned into the pCRII-TOPO vector using the TOPO™ cloning system (Invitrogen), according to manufacturer's standard protocol. The TOPO cloning system is a variation of the TA cloning system allowing the rapid cloning of PCR products, taking advantage from the fact that Taq polymerase leaves a single Adenosine at the 3′ end of PCR products. Since the TOPO vector has single-stranded Thymine overhangs, Topoisomerase I enzyme is able to join the T-ends of the vector to the A-overhangs of the PCR product, which can be used without any purification step.

The resulting plasmids (pCRTOPO-ORF series) were used to transform E. coli cells (TOP10F′, Invitrogen, supplied with the TOPO TA Cloning Kit), obtaining several clones for each ORF. Plasmid DNA was isolated using a commercial kit (WIZARD Plasmid Minipreps; Promega) and sequenced to verify the identity of the amplified and cloned sequence with the originally selected human genomic DNA sequence.

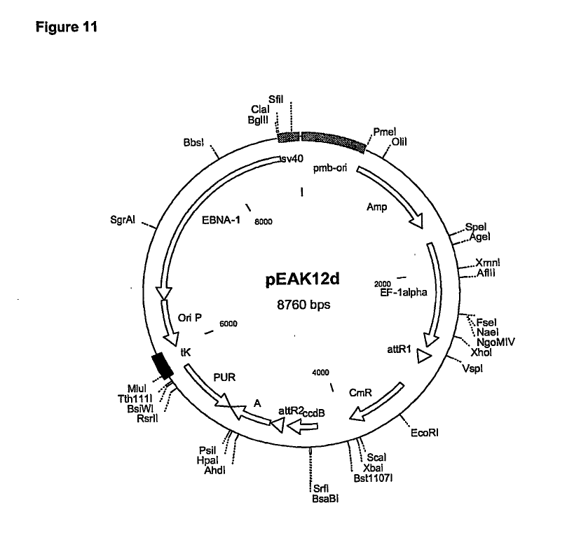

The plasmids containing the desired sequences were used in a further round of PCR reactions necessary for transferring the ORFs into the expression vector pEAK12d (FIG. 11), which allows the expression of the cloned insert under the control of EF-1α promoter and in frame with a 6-Histidine Tag sequence, using the Gateway cloning system (Invitrogen).

The expression vector pEAK12D was constructed by modifying pEAK12 (Edge Biosystems). This vector was digested with HindIII and NotI, made blunt ended with Klenow and dephosphorylated using calf-intestinal alkaline phosphatase. After dephosphorylation, the vector was ligated to blunt ended Gateway reading frame cassette C (Gateway vector conversion system, Invitrogen cat no. 11828-019) that contains AttR recombination sites flanking the ccdB gene (marker for negative selction of non-recombinant plasmids) and chloramphenicol resistance. The resulting plasmids were used to transform DB3.1 E. coli cells, which allow propagation of vectors containing the ccdB gene. Miniprep DNA was isolated from several of the resultant colonies and digested with AseI/EcoRI to identify clones yielding a 670 bp fragment, obtainable only when the cassette had been inserted in the correct orientation. The resultant plasmid was called pEAK12D.

Two series of primers (Table IV) were designed to add the ATTB1 and ATTB2 recombination sites (necessary for the integration in the expression vector) at the 5′ and 3′ end, respectively, of the ORF-containing insert. In the first series of primers (EX1 series), the original ORF-specific CL primers were modified by adding, at the 5′ end, the sequence AAGCAGGCTTCGCCACC (for forward primers) or GTGATGGTGATGGTG (for reverse primers, but after eliminating the nucleotides complementary to the stop codon). In the second series of primers (EX2 series), the original ORF-specific CL primers were modified by adding, at the 5′ end, the sequence GGGGACAAGTTTGTACAAAAAAGCAGGCTTCGCCACC (for forward primers) or GGGGACCACTTTGTACAAGAAAGCTGGGTTTCAATGGTGATGGTGATGGTG (for reverse primers, but after eliminating the nucleotides complementary to the stop codon). These reverse primers contain the codons for the 6-Histidine tag, which then results fused in frame with the ORFs at their C-terminal end.

The PCR amplification was performed in 2 consecutive reactions. The first one was performed by mixing the following components (total volume 50 μl in double-distilled water):
25 ng pCRTOPO-ORF vector
5 mM dNTP (Invitrogen)
0.5 μl Pfx DNA polymerase (Invitrogen)
0.5 μl each EX1 primer (100 μM)
5 μl Pfx polymerase buffer 10× (Invitrogen)

The PCR reactions were performed using an initial denaturing step of 95° C. for 2 minutes, followed by 10 cycles:
94° C. for 15 seconds
68° C. for 30 seconds

The PCR products were purified using the Wizard PCR prep DNA purification system (Promega), and added as templates in a second PCR reaction including the following components (total volume 50 μl In double-distilled water):
10 μl purified PCR product
5 mM dNTP (Invitrogen)
0.5 μl Pfx DNA polymerase (Invitrogen)
0.5 μl each EX2 primer (100 μM)
5 μl Pfx polymerase buffer 10× (Invitrogen)

The PCR reactions were performed an initial denaturing step of 95° C. for 1 minute, followed by 4 cycles:
94° C. for 15 seconds
50° C. for 30 seconds
68° C. for 3 minutes 30 seconds

Then the following conditions were applied for 25 cycles:
94° C. for 15 seconds
55° C. for 30 seconds
68° C. for 3 minutes 30 seconds.

The DNA fragments resulting from the PCR reactions were purified as described before and recombined into the pEAK12d vector using the Gateway system.

First, the following 10 μl reactions were assembled:

| | | |
|---|---|---|
| pDONR-201 (0.1 μg/μl) | 1.5 | μl |
| PCR product | 5 | μl |
| BP buffer | 2 | μl |
| BP enzyme mix | 1.5 | μl |

After being incubated at room temperature for 1 hour, the reaction was stopped by adding proteinase K (1 μl, 2 μg) and incubating at 37° C. for further 10 minutes.

An aliquot of this reaction (2 μl) was used for transforming E. coli cells (strain DH10B) by electroporation. Plasmid DNA was prepared for 4 clones for each ORF and used for parallel 10 μl recombination reactions containing:

| | | |
|---|---|---|
| pEAK12d (0.1 μg/μl) | 1.5 | μl |
| Plasmid DNA | 1.5 | μl |
| ddH20 | 3.5 | μl |
| LR buffer | 2 | μl |
| LR enzyme mix | 1.5 | μl |

After being incubated at room temperature for 1 hour, the reaction was stopped by adding proteinase K (1 μl, 2 μg) and incubating at 37° C. for further 10 minutes. An aliquot of this reaction (1 μl) was used for transforming DH10B E. coli cells by electroporation. The clones containing the correct insert were identified first by performing colony PCR on 3 colonies using the forward and reverse vector primers pEAK12d F1 (GCCAGCTTGGCACTTGATGT) and pEAK12d R1 (GATGGAGGTGGACGTGTCAG), then confirmed by sequencing the insert with the same primer.

### Example 3

### Expression and Purification of the 6-Histine-Tagged Chemokine-Like Polypeptides in Mammalian Cells

Human Embryonic Kidney cells expressing the Epstein-Barr virus Nuclear Antigen (HEK293-EBNA) were seeded in T225 flasks (50 ml at a density of 2×10⁵ cells/ml) from 16 to 20 hours prior to transfection, which was performed using the cationic polymer reagent JetPEI™ (PolyPlus-transfection; 2 μl/μg of plasmid DNA). For each flask, 113 μg of the ORF-specific pEAK12d plasmid, which were prepared using CsCl (Sambrook, J et al. “Molecular Cloning, a laboratory manual”; 2nd edition. 1989; Cold Spring Harbor Laboratory Press), were co-transfected with 2.3 μg of a plasmid acting as positive control since it expresses Green Fluorescent Protein (GFP). The plasmids, diluted in 230 μl of JetPEI™ solution, were added to 4.6 ml of NaCl 150 mM, vortexed and incubated for 30 minutes at room temperature. This transfection mix was then added to the T225 flask and incubated at 37° C. for 6 days. An aliquot of the cultures was then exposed to UV irradiation to check the transfection efficiency by evaluating GFP fluorescence.

Culture medium from HEK293-EBNA cells transfected with the same plasmids were pooled and 100 ml of the medium were diluted to 200 ml with 100 ml of ice-cold buffer A (50 mM NaH₂PO₄; 600 mM NaCl; 8.7% (w/v) glycerol, pH 7.5), which is the same buffer used for equilibrating the affinity column on which His-tagged proteins were subsequently immobilized and eluted. The solution was filtered through a 0.22 μm sterile filter (Millipore) and kept at 4° C. in 250 ml sterile square media bottles until further processing.

Two consecutive chromatography procedures were applied to the samples at 4° C. using an HPLC-based system (Perfusion Chromatography™, PerSeptive Biosystems) including a VISION workstation (BioCAD™ series), POROS™ chromatographic media, and an external 250 ml-sample loader (Labomatic).

In the first chromatography step, a Ni-metal affinity column (0.83 ml, POROS 20 MC) was first regenerated with 30 column volumes of EDTA solution (100 mM EDTA; 1 M NaCl; pH 8.0), and then recharged with Ni ions through washing with 15 column volumes of the Ni solution (100 mM NiSO₄). The column is subsequently washed with 10 column volumes of buffer A, 7 column volumes of buffer B (50 mM.NaH₂PO₄; 600 mM NaCl; 8.7% (w/v) glycerol, 400 mM; imidazole, pH 7.5), and finally equilibrated with 15 column volumes of buffer A containing 15 mM imidazole. The sample loader charged the protein-containing solution onto the Ni metal affinity column at a flow rate of 10 ml/min. The column was then washed with 12 column volumes of Buffer A, followed by 28 column volumes of Buffer A containing a concentration of imidazole (20 mM) allowing the elution of contaminating proteins that are loosely attached to the Ni-column. The His-tagged protein is finally eluted with 10 column volumes of Buffer B at a flow rate of 2 ml/min, collecting collected 1.6 ml fractions.

In the second chromatography step, a gel-filtration column (10 ml G-25 5Sephadex) was regenerated with 2 ml of buffer D (137 mM NaCl; 2.7 mM KCl; 1.5 mM KH₂PO₄; 8 mM Na₂HPO₄; 1 M NaCl; pH 7.2), and then equilibrated with 2 column volumes of buffer C (137 mM NaCl; 2.7 mM KCl; 1.5 mM KH₂PO₄; 8 mM Na₂HPO₄; 20% (w/v) glycerol; pH 7.4) before injecting the Ni-column peak fractions onto this column. The sample is eluted with buffer C and the desalted sample is recovered in 2.2 ml fractions. The peak fractions from the gel-filtration column were filtered through a 0.22 μm sterile centrifugation filter (Millipore) and aliquots (20 μl) were analyzed in parallel on SDS-PAGE (4-12% NuPAGE gel; Novex) by Coomassie staining and by Western blot with antibodies recognizing Histidine tags. Protein concentrations were determined in the samples that show detectable protein bands by Coomassie staining, using the BCA Protein Assay kit (Pierce) and Bovine Serum Albumin as standard.

The gel for the Western blot analysis was electrotransferred to a nitrocellulose membrane at 290 mA at 4° C. for 1 hour. The membrane is blocked with 5% milk powder in PBS (137 mM NaCl; 2.7 mM KCl; 1.5 mM KH₂PO₄; 8 mM Na₂HPO₄;pH 7.4), and subsequently incubated with a mixture of 2 rabbit polyclonal anti-Histidine tag antibodies (G-18 and H-15, 0.2 μg/ml each; Santa Cruz) at 4° C. overnight. After a further 1 hour incubation at room temperature, the membrane was washed with PBS containing 0.1% Tween-20 (3×10 min), and then exposed to a secondary HRP-conjugated anti-rabbit antibody (DAKO) at room temperature for 2 hours. After washing in PBS containing 0.1% Tween-20 (3×10 minutes), the ECL kit (Amersham Pharmacia) was used to detect the antibodies immobilized onto the membrane, comparing the film with the image of the Coomassie stained gel.

### Example 4

### Cell- and Animal-Based Assay for the Validation and Characterization of the Chemokine-Like Polypeptides

Several assays have been developed for testing specificity, potency, and efficacy of chemokines using cell cultures or animal models, for example in vitro chemotaxis assays (Proudfoot A E et al., 2001; Lust-Narasimhan M et al., 1995), or mouse ear swelling (Garrigue J L et al., 1994). Many other assays and technologies for generating useful tools and products (antibodies, transgenic animals, radiolabeled proteins, etc.) have been described in reviews and books dedicated to chemokines (Methods Mol. Biol vol. 138, “Chemokines Protocols”, edited by Proudfoot A I et al., Humana Press Inc., 2000; Methods Enzymol, vol. 287 and 288, Academic Press, 1997), and can be used to verify, in a more precise manner, the biological activities of the chemokine-like polypeptides of the invention and related reagents in connection with possible therapeutic or diagnostic methods and uses.

TABLE I

| | | More Preferred Synonymous |
|---|---|---|
| Amino Acid | Synonymous Groups | Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

TABLE II

| Amino Acid | Synonymous Groups |
|---|---|
| Ser | D-Ser, Thr, D-Thr, allo-Thr, Met, D-Met, Met(O), |
| | D-Met(O), L-Cys, D-Cys |
| Arg | D-Arg, Lys, D-Lys, homo-Arg, D-homo-Arg, Met, Ile, |
| | D-.Met, D-Ile, Orn, D-Orn |
| Leu | D-Leu, Val, D-Val, AdaA, AdaG, Leu, D-Leu, Met, D-Met |
| Pro | D-Pro, L-I-thioazolidine-4-carboxylic acid, D-or |
| | L-1-oxazolidine-4-carboxylic acid |
| Thr | D-Thr, Ser, D-Ser, allo-Thr, Met, D-Met, Met(O), |
| | D-Met(O), Val, D-Val |
| Ala | D-Ala, Gly, Aib, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Aib, .beta.-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, |
| | Trans-3,4, or 5-phenylproline, AdaA, AdaG, cis-3,4, |
| | or 5-phenylproline, Bpa, D-Bpa |
| Tyr | D-Tyr, Phe, D-Phe, L-Dopa, His, D-His |
| Cys | D-Cys, S-Me-Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, D-Met, |
| | Ile, D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S—Me--Cys, Ile, D-Ile, Leu, D-Leu, Val, D-Val |

TABLE III

| SEQ | | | |
|---|---|---|---|
| ID | | | |
| NO: | NAME | DIRECTION | 5′→3′ SEQUENCE |
| 17 | CL_1754_5 | Forward | ATGAATGTCATTGTTTTACA |
| 18 | CL_1754_3 | Reverse | CTACCAACCTGTACAGCATG |
| 19 | CL_4922_5 | Forward | CTGACTATGATGAGGGTGCT AAGGC |
| 20 | CL_4922_3 | Reverse | TCAAATTGCTGGGAAAGTTC TCAGG |
| 21 | CL_5008_5 | Forward | CATGATCTTTGGCCTGCTAA TC |
| 22 | CL_5008_3 | Reverse | TTAAAGGGAAAGTAATAGGA G |
| 23 | CL_0210_5 | Forward | CTATGGGCTTTGTTGTTCTA TG |
| 24 | CL_0210_3 | Reverse | TCAGAAAAATTCTAACAAAA TTG |
| 25 | CL_0711_5 | Forward | ATGGTTATTCCACATCTTG |
| 26 | CL_0711_3 | Reverse | TCATCTCTGTTGCAGCAAAC G |
| 27 | CL_4320_5 | Forward | ATGTTATTTACTTTATTATT C |
| 28 | CL_4320_3 | Reverse | TCACAGAAAAATCAAAGAGG |

TABLE IV

| SEQ | | | |
|---|---|---|---|
| ID | | | |
| NO: | NAME | DIRECTION | 5′→3′ SEQUENCE |
| 29 | EX1_1754_5 | Forward | AAGCAGGCTTCGCCACCATGAATGTC |
| | | | ATTGTTTTACA |
| 30 | EX1_1754_3 | Reverse | GTGATGGTGATGGTGCCAACCTGTAC |
| | | | AGCATG |
| 31 | EX1_4922_5 | Forward | AAGCAGGCTTCGCCACCCTGACTATG |
| | | | ATGAGGGTGCT AAGGC |
| 32 | EX1_4922_3 | Reverse | GTGATGGTGATGGTGAATTGCTGGGA |
| | | | AAGTTC TCAGG |
| 33 | EX1_5008_5 | Forward | AAGCAGGCTTCGCCACC CATGATCT |
| | | | TTGGCCTGCTAA TC |
| 34 | EX1_5008_3 | Reverse | GTGATGGTGATGGTGAAGGGAAAGTA |
| | | | ATAGGA G |
| 35 | EX1_0210_5 | Forward | AAGCAGGCTTCGCCACCCTATGGGCT |
| | | | TTGTTGTTCTA TG |
| 36 | EX1_0210_3 | Reverse | GTGATGGTGATGGTGGAAAAATTCTA |
| | | | ACAAAA TTG |
| 37 | EX1_0711_5 | Forward | AAGCAGGCTTCGCCACCATGGTTATT |
| | | | CCACATCTTG |
| 38 | EX1_0711_3 | Reverse | GTGATGGTGATGGTGTCTCTGTTGCA |
| | | | GCAAACG |
| 39 | EX1_4320_5 | Forward | AAGCAGGCTTCGCCACCATGTTATTT |
| | | | ACTTTATTATTC |
| 40 | EX1_4320_3 | Reverse | GTGAEGGTGATGGTGCAGAAAAATCA |
| | | | AAGAGG |
| 41 | EX2_1754_5 | Forward | GGGGACAAGTTTGTACAAAAAAGCAG |
| | | | GCTTCGCCACCATGAATGTC ATTGT |
| | | | TTTACA |
| 42 | EX2_1754_3 | Reverse | GGGGACCACTTTGTACAAGAAAGCTG |
| | | | GGTTTCAATGGTGATGGTGATGGTGC |
| | | | CAACCT GTACAGC |
| 43 | EX2_4922_5 | Forward | GGGGACAAGTTTGTACAAAAAAGCAG |
| | | | GCTTCGCCACC CTGACTATGATGAG |
| | | | GGTGCTAA |
| 44 | EX2_4922_3 | Reverse | GGGGACCACTTTGTACAAGAAAGCTG |
| | | | GGTTTCAATGGTGATGGTGATGGTGA |
| | | | ATTGCTCGGAAAGTTC TC |
| 45 | EX2_5008_5 | Forward | GGGGACAAGTTTGTACAAAAAAGCAG |
| | | | GCTTCGCCACC CATGATCTTTGGCC |
| | | | TGCTAATC |
| 46 | EX2_5008_3 | Reverse | GGGGACCACTTTGTACAAGAAAGCTG |
| | | | GGTTTCAATGGTGATGGTGATGGTGA |
| | | | AGGGAAAGTAATAGGAG |
| 47 | EX2_0210_5 | Forward | GGGGACAAGTTTGTACAAAAAAGCAG |
| | | | GCTTCGCCACC CTATGGGCTTTGTT |
| | | | GTTCT |
| 48 | EX2_0210_3 | Reverse | GGGGACCACTTTGTACAAGAAAGCTG |
| | | | GGTTTCAATGGTGATGGTGATGGTGG |
| | | | AAAAATTCTAACAAAA |
| 49 | EX2_0711_5 | Forward | GGGGACAAGTTTGTACAAAAAAGCAG |
| | | | GCTTCGCCACC ATGGTTATTCCACA |
| | | | TCTTG |
| 50 | EX2_0711_3 | Reverse | GGGGACCACTTTGTACAAGAAAGCTG |
| | | | GGTTTCAATGGTGATGGTGATGGTGT |
| | | | CTCTGTTGCAGCAAAC G |
| 51 | EX2_4320_5 | Forward | GGGGACAAGTTTGTACAAAAAAGCAG |
| | | | GCTTCGCCACC ATGTTATTTACTTT |
| | | | ATTATTC |
| 52 | EX2_4320_3 | Reverse | GGGGACCACTTTGTACAAGAAAGCTG |
| | | | GGTTTCAATGGTGATG GTGATGGTG |
| | | | CAGAAAAATCAAAGAGG |

### REFERENCES

Altschul S F et al., J Mol Biol, 215: 403-10, 1990.
Andersen D C and Krummen L, Curr Opin Biotechnol, 13: 117-23, 2002.
Baggiolini M et al., Annu Rev Immunol, 15: 675-705, 1997.
Baggiolini M, J Intem Med, 250: 91-104, 2001.
Baker K N et al., Trends Biotechnol, 20: 149-56, 2002.
Blagoev B and Pandey A, Trends Biochem Sci, 26: 639 -41, 2001.
Bock A, Science, 292: 453-4, 2001.
Brown A et al., J Pept Sci, 2: 40-46, 1996.
Browne M J, J Biotechnol, 78:247-250, 2000.
Bunz F, Curr Opin Oncol, 14: 73-8, 2002.
Burgess R R and Thompson N E, Curr Opin Biotechnol, 12: 450-4, 2001.
Chambers S P, Drug Disc Today, 14: 759-765, 2002.
Chantry D F et al., J Leukoc Biol, 64: 49-54, 1998.
Choy E H and Panayi G S, N Engl J Med, 344: 907-16, 2001.
Chu L and Robinson D K, Curr Opin Biotechnol, 13: 304-8, 2001.
Cleland J L et al., Curr Opin Biotechnol, 12: 212-9, 2001.
Coleman T A et al., Gene 190: 163-171, 1997.
Coleman R A et al., Drug Discov Today, 6: 1116-1126, 2001.
Constans A, The Scientist, 16: 37, 2002.
Davis B G and Robinson M A, Curr Opin Drug Discov Devel, 5: 279-88, 2002.
Dower S K, Nat Immunol, 1: 367-8, 2000.
Dougherty D A, Curr Opin Chem Biol, 4: 645-52, 2000.
Ehlert J E et al., J Immunol, 161: 4975-4982, 1998.
Fernandez E J and Lolis E, Annu Rev Pharmacol Toxicol, 42: 469-499, 2002.
Frederick M J and Clayman G L, Exp Rev Mol Med 2001, http://www-ermm.cbcu.cam.ac.uk/reviews.htm.
Garnett M C, Adv Drug Deliv Rev, 53: 171-216, 2001.
Garrigue J L et al., Contact Dermattis, 30: 231-7, 1994.
Gavilondo J V and Larrick J W, Biotechniques, 29: 128-136, 2000.
Gendel S M, Ann N Y Acad Sci, 964: 87-98, 2002.
Giddings G, Curr Opin Biotechnol, 12: 450-4, 2001.
Gish W and States D J, Nat Genet, 3: 266-72, 1993.
Godessart N and Kunkel S L, Curr Opin Immunol, 13: 670-675, 2001.
Goleblowski A et al., Curr Opin Drug Discov Devel, 4: 428-34, 2001.
Gupta P et al., Drug Discov Today, 7: 569-579, 2002.
Haskell C A et al., Curr Opin Invest Drugs, 3: 399-455, 2002.
Haupt K, Nat Biotechnol, 20: 884-885, 2002.
Hruby V J and Balse P M, Curr Med Chem, 7: 945-70, 2000.
Johnson D E and Wolfgang G H, Drug Discov Today, 5: 445-454, 2000.
Kane J F, Curr Opin Biotechnol, 6: 494-500, 1995.
Kolb A F, Cloning Stem Cells, 4: 65-80, 2002.
Kuroiwa Y et al., Nat Biotechnol, 20: 889-94, 2002.
Lambeir A, et al. J Biol Chem, 276: 29839-29845, 2001.
Lewis D L, Nat Genet, 32: 107-8, 2002.
Li A P, Drug Discov Today, 6: 357-366, 2001.
Lin Cereghino G P et al., Curr Opin Biotechnol, 13: 329-332, 2001.
Loetscher P and Clark-Lewis I, J Leukoc Biol, 69: 881-884, 2001.
Lowe C R et al., J Biochem Biophys Methods, 49: 561-74, 2001.
Lucas A D and Greaves D R, Exp Rev Mol Med 2001, http://www-ermm.cbcu.cam.ac.uk/reviews.htm.
Luo B and Prestwich G D, Exp Opin Ther Patents, 11: 1395-1410, 2001.
Lusti-Narasimhan M et al., J Biol Chem, 270: 2716-21, 1995.
Mulder N J and Apweiler R, Genome Biol, 3(1): REVIEWS2001, 2002
Murphy L R et al., Protein Eng, 13: 149-52, 2000.
Nilsson J et al., Protein Expr Purif, 11: 1-16, 1997.
Nomiyama H et al., Genes Immun, 2: 110-113, 2001.
Paddison P J, Proc Nati Acad Sci USA, 99: 1443-8, 2002.
earson W R and Miller W, Methods Enzymol, 210: 575-601, 1992.
Pellois J P et al., Nat Biotechnol, 20: 922-6, 2002.
Pillai O and Panchagnula R, Cur Opin Chem Biol, 5: 447-451, 2001
Proost P, et al. Blood, 98: 3554-3561, 2001.
Proudfoot A., Immunol Rev, 177: 246-256, 2000.
Proudfoot A et al., J Biol Chem 276: 10620-10626, 2001.
Quinn-Senger K E et al., Curr Opin Chem Biol, 6: 418-26, 2002.
Reape T J and Groot P H, Atherosclerosis, 147: 213-25, 1999.
Rehm B H, Appl Microbiol Biotechnol, 57: 579-92, 2001.
Robinson C R, Nat Biotechnol, 20: 879-880, 2002.
Rogov S I and Nekrasov A N, Protein Eng, 14: 459-463, 2001.
Rossi D et al., J.Immunol, 158: 1033-1036, 1997.
Rossi D and Ziotnik A, Annu Rev Immunol, 18: 217-42, 2000.
Schellekens H, Nat Rev Drug Discov, 1: 457-62, 2002
Schwarz M K and Wells T N, Curr Opin Chem Biol, 3: 407-17, 1999.
Shelbani N, Prep Biochem Biotechnol, 29: 77-90, 1999.
Stein C A, J Clin Invest, 108: 641-4, 2001.
Stein L D, Methods Biochem Anal, 43: 413-49, 2001.
Stevanovic S, Nat Rev Cancer, 2: 514-20, 2002.
Templin M F et al., Trends Biotechnol, 20: 160-6, 2002.
Tribbick G, J Immunol Methods, 267: 27-35, 2002.
van den Burg B and Eijsink V, Curr Opin Biotechnol, 13: 333-337, 2002.
van Dijk M A and van de Winkel J G, Curr Opin Chem Biol, 5: 368-74, 2001.
Villain M et al., Chem Biol, 8: 673-9, 2001.
Wells T N and Peitsch M C. Methods Mol Biol, 138: 65-73, 2000.
Yoshie O F et al., Adv Immunol, 78: 57-110, 2001.

1.-41. (canceled)

42. A composition of matter comprising:
a) an isolated polypeptide having chemotactic activity selected from the group consisting of:
1) the amino acid sequences SEQ ID NO: 2, 4, 6, 8, 10, 12, 14, or 16;
2) the mature form of the polypeptides SEQ ID NO: 2, 4, 6, 8, 10, 12, 14, or 16;
3) the polypeptides comprising the Cysteine-rich region of SEQ ID NO: 2, 4, 6, 8, 10, 12, 14, or 16;
4) the active variants of the amino acid sequence given by SEQ ID NO: 2, 4, 6, 8, 10, 12, 14, or 16 wherein any amino acid specified in the chosen sequence is non-conservatively substituted, provided that no more than 15% of the amino acid residues in the sequence are so changed;
5) the active fragments, precursors, salts, or derivatives of the amino acid sequences given in 1) to 4);
6) a naturally occurring allelic variant of the sequence given by SEQ ID NO: 2, 4, 6, 8, 10, 12, 14, or 16;
7) a naturally occurring allelic variant of the sequence given by SEQ ID NO: 2, 4, 6, 8, 10, 12, 14, or 16, wherein the variant is the translation of a single nucleotide polymorphism;
8) a polypeptide of any of 1) to 7), wherein the polypeptide binds specifically an antibody or a binding protein generated against SEQ ID NO: 2, 4, 6, 8, 10, 12, 14, or 16 or a fragment thereof;
9) a fusion protein comprising a polypeptide according to any of 1) to 8);
10) a fusion protein comprising a polypeptide according to any of 1) to 8), wherein said proteins further comprise one or more amino acid sequence belonging to these protein sequences: membrane-bound protein, immunoglobulin constant region, multimerization domains, extracellular proteins, signal peptide-containing proteins, export signal-containing proteins;
11) a polypeptide encoded by a nucleic acid that hybridizes under high stringency conditions with a nucleic acid selected from the group consisting of SEQ ID NO: 1, 3, 5, 7, 9, 11, 13, or 15, or a complement of said DNA sequences or exhibits at least about 85% identity over a stretch of at least about 30 nucleotides, with a nucleic acid selected from the group consisting of SEQ ID NO: 1, 3, 5, 7, 9, 11, 13, or 15, or a complement of said DNA sequences;
b) an antagonist of a polypeptide of a1) to a8), wherein said antagonist comprises an amino acid sequence resulting from the modification of one or more residues of said polypeptide;
c) a ligand:
1) that binds specifically to a polypeptide according to any one of a1) to a8);
2) that binds specifically to a polypeptide according to any one of a1) to a8) and that antagonizes or inhibits the chemotactic activity of a polypeptide according to any one of a1) to a8);
3) which is a monoclonal antibody, a polyclonal antibody, a humanized antibody, an antigen binding fragment, or the extracellular domain of a membrane-bound protein;
d) an isolated nucleic acid:
1) encoding polypeptides of any of a1) to a8), said polypeptides having chemotactic activity;
2) encoding the fusion proteins of a9) or a10);
3) encoding the antagonists of b);
4) comprising a DNA sequence selected from the group consisting of SEQ ID NO: 1, 3, 5, 7, 9, 11, 13, or 15, or the complement of said DNA sequences;
5) that hybridizes under high stringency conditions with a nucleic acid selected from the group consisting of SEQ ID NO: 1, 3, 5, 7, 9, 11, 13, or 15, or a complement of said DNA sequences; or
6) exhibiting at least about 85% identity over a stretch of at least about 30 nucleotides, with a nucleic acid selected from the group consisting of SEQ ID NO: 1, 3, 5, 7, 9, 11, 13, or 15, or a complement of said DNA sequences;
e) a vector comprising a nucleic acid according to d);
f) a peptide mimetic designed on the sequence or the structure or both the sequence and structure of a polypeptide according to any one of a1) to a8);
g) a host cell transformed with a vector or a nucleic acid according to any of d) or e);
h) a primer comprising SEQ ID NO: 17, 18, 19, 20, 21, 22, 23, 24, 25, 26, 27, or 28;
i) a compound that enhances the expression level of a polypeptide according to any one a1) to a8);
j) a compound that reduces the expression level of a polypeptide according to any one a1) to a8);
k) a pharmaceutical composition comprising any one of a) to j) and a pharmaceutically acceptable carrier;
l) a kit for measuring the activity and/or the presence of a chemokine-like polypeptide comprising one or more of the following reagents set forth in a) to k);
m) a transgenic animal cell that has been transformed with a vector or a nucleic acid according to d) or e), having constitutive or inducible altered expression levels of a polypeptide according to any one of a1) to a8); or
n) a transgenic non-human animal that has been transformed to have enhanced or reduced expression levels of a polypeptide according to a1) to a8).

43. The composition of matter of claim 42, wherein the nucleic acid molecule in said vector is operatively linked to expression control sequences allowing expression in prokaryotic or eukaryotic host cells of the encoded polypeptide.

44. The composition of matter of claim 42, wherein said polypeptides or ligands are in the form of active conjugates or complexes with a molecule chosen from radioactive labels, fluorescent labels, biotin, or cytotoxic agents.

45. The composition of matter of claim 42, wherein the compound that reduces the expression level of a polypeptide is an antisense oligonucleotide or a small interfering RNA.

46. A process for producing cells that produce a polypeptide comprising transforming a cell with a polynucleotide selected encoding SEQ ID NO: 2, 4, 6, 8, 10, 12, 14, or 16 and culturing said cells to produce a polypeptide.

47. A method of using a composition of matter comprising obtaining a composition of matter according to claim 42 and using said composition of matter in a method selected from: making a genetically engineered cell; producing a polypeptide; producing a pharmaceutical composition; the treatment of a disease; screening candidate compounds; identifying a candidate compound; or determining the activity, presence or both activity or presence of said composition of matter.

48. The method of claim 47, comprising genetically engineering cells with composition of matter comprising a vector or a nucleic acid according to claim 42.

49. The method of claim 48, further comprising culturing said cell under conditions in which the nucleic acid or vector is expressed, and recovering the polypeptide encoded by said nucleic acid or vector from the culture.

50. The method of claim 47, comprising the administration of a therapeutically effective amount of a composition of matter comprising a polypeptide, a ligand, an antagonist, a peptide mimetic, a nucleic acid, a cell, or a compound as set forth in claim 42 to an individual for the treatment of a disease.

51. The method of claim 47, comprising a composition of matter comprising a polypeptide, a ligand, an antagonist, a peptide mimetic, a nucleic acid, a cell, or a compound as set forth in claim 42 with a pharmaceutically acceptable carrier to produce a pharmaceutical composition.

52. The method of claim 47, wherein said method screens for candidate compounds effective to treat a disease related to the chemokine-like polypeptides and comprises:
a) contacting a cell, a transgenic animal cell, or a transgenic non-human animal as set forth in claim 42 and having enhanced or reduced expression levels of the polypeptide with a candidate compound; and
b) determining the effect of the compound on the animal or on the cell.

53. The method of claim 47, wherein said method identifies a candidate compound as an antagonist/inhibitor or agonist/activator of a polypeptide of claim 42 and comprises:
a) contacting said polypeptide of claim 42, said compound, and a mammalian cell or a mammalian cell membrane capable of binding the polypeptide; and
b) measuring whether the molecule blocks or enhances the interaction of the polypeptide, or the response that results from such interaction, with the mammalian cell or the mammalian cell membrane.

54. The method of claim 47, said method determining the activity and/or the presence of a polypeptide in a sample, the method comprising:
a) providing a protein-containing sample;
b) contacting said sample with a ligand of claim 1; and
c) determining the presence of said ligand bound to said polypeptide.

55. The method of claim 47, said method determining the presence or the amount of a transcript or of a nucleic acid encoding a polypeptide and comprising:
a) providing a nucleic acid-containing sample;
b) contacting said sample with a nucleic acid of claim 42; and
determining the hybridization of said nucleic acid with a nucleic acid into the sample.

56. The method of claim 55, wherin said contacting comprises the use of the primer sequences comprising SEQ ID NO: 17, 18, 19, 20, 21, 22, 23, 24, 25, 26, 27 or 28 for determining the presence or the amount of a transcript or of a nucleic acid encoding the polypeptide.

57. The method of claim 56, wherein the amount of said transcript or nucleic acid is determined by Polymerase Chain Reaction.
